(19) United States
(12) Patent Application Publication
(10) Pub. No.: US 2002/0160416 A1
(43) Pub. Date: Oct. 31, 2002

(54) Receptor from TNF family

(76) Inventors: William J. Boyle, Moorpark, CA (US); Hailing Hsu, Moorpark, CA (US)

Correspondence Address: AMGEN INC., U.S. Patent Operations/ TJG, Dept. 4300, M/S 27-4-A, One Amgen Center Drive, Thousand Oaks, CA, 91320-1799, US

(21) Appl. No.: 09/779,050

(22) Filed: Feb. 12, 2001

Related U.S. Application Data
(60) Provisional application No. 60/181,800, filed on Feb. 11, 2000.

(51) Int. Cl. G01N 33/53; C07K 16/46; C08B 37/00

(52) U.S. Cl. 435/7.1; 530/389.1; 530/395; 536/53

(57) ABSTRACT

A member of the tumor necrosis factor family and its receptor are described. This member is primarily expressed in B cells and its expression correlates to increases in the number of B cells and immunoglobulins produced. The natural, preferred human ortholog is here called AGP-3R. The protein is a type III transmembrane protein and has an amino terminal extracellular domain, a transmembrane domain, and a carboxy terminal intracellular domain. AGP-3R-related proteins of the invention may be membrane-associated or in soluble form, recombinantly produced or isolated after natural production. The invention provides for nucleic acids encoding such AGP-3R-related proteins, vectors and host cells expressing the polypeptides, and methods for producing recombinant proteins. Antibodies or fragments thereof that specifically bind the proteins are also provided.

This application claims the benefit of U.S. Provisional Application Serial No. 60/181,800, filed Feb. 11, 2000, which is hereby incorporated by reference.

### FIELD OF THE INVENTION

The present invention relates to proteins that are involved in inflammation and immunomodulation, particularly in B cell growth, survival, or activation. The invention further relates to proteins related to the tumor necrosis factor (TNF)/nerve growth factor (NGF) superfamily and related nucleic acids, expression vectors, host cells, and binding assays. The specification also describes compositions and methods for the treatment of immune-related and inflammatory, autoimmune and other immune-related diseases or disorders, such as rheumatoid arthritis (RA), Crohn's disease (CD), lupus, and graft versus host disease (GvHD).

### BACKGROUND OF THE INVENTION

After years of study in necrosis of tumors, tumor necrosis factors (TNFs) α and β were finally cloned in 1984. The ensuing years witnessed the emergence of a superfamily of TNF cytokines, including fas ligand (FasL), CD27 ligand (CD27L), CD30 ligand (CD30L), CD40 ligand (CD40L), TNF-related apoptosis-inducing ligand (TRAIL, also designated AGP-1), osteoprotegerin binding protein (OPG-BP or OPG ligand), 4-1BB ligand, LIGHT, APRIL, and TALL-1. Smith et al. (1994), Cell 76: 959-962; Lacey et al. (1998), Cell 93: 165-176; Chichepotiche et al. (1997), J. Biol. Chem. 272: 32401-32410; Mauri et al. (1998), Immunity 8: 21-30; Hahne et al. (1998), J.Exp. Med. 188: 1185-90; Shu et al. (1999), J. Leukocyte Biology 65: 680-3. This family is unified by its structure, particularly at the C-terminus. In addition, most members known to date are expressed in immune compartments, although some members are also expressed in other tissues or organs, as well. Smith et al. (1994), Cell 76: 959-62. All ligand members, with the exception of LT-α, are type II transmembrane proteins, characterized by a conserved 150 amino acid region within C-terminal extracellular domain. Though restricted to only 20-25% identity, the conserved 150 amino acid domain folds into a characteristic β-pleated sheet sandwich and trimerizes. This conserved region can be proteolyticaly released, thus generating a soluble functional form. Banner et al. (1993), Cell 73: 431-445.

Many members within this ligand family are expressed in lymphoid enriched tissues and play important roles in the immune system development and modulation. Smith et al. (1994). For example, TNFα is mainly synthesized by macrophages and is an important mediator for inflammatory responses and immune defenses. Tracey & Cerami (1994), Annu. Rev. Med. 45: 491-503. Fas-L, predominantly expressed in activated T cell, modulates TCR-mediated apoptosis of thymocyts. Nagata, S. & Suda, T. (1995) Immunology Today 16: 39-43; Castrim et al. (1996), Immunity 5: 617-27. CD40L, also expressed by activated T cells, provides an essential signal for B cell survival, proliferation and immunoglobulin isotype switching. Noelle (1996), Immunity 4: 415-9.

The cognate receptors for most of the TNF ligand family members have been identified. These receptors share characteristic multiple cysteine-rich repeats within their extracellular domains, and do not possess catalytic motifs within cytoplasmic regions. Smith et al. (1994). The receptors signal through direct interactions with death domain proteins (e.g. TRADD, FADD, and RIP) or with the TRAF proteins (e.g. TRAF2, TRAF3, TRAF5, and TRAF6), triggering divergent and overlapping signaling pathways, e.g. apoptosis, NF-κB activation, or JNK activation. Wallach et al. (1999), Annual Review of Immunology 17: 331-67. These signaling events lead to cell death, proliferation, activation or differentiation. The expression profile of each receptor member varies. For example, TNFR1 is expressed on a broad spectrum of tissues and cells (13); whereas the cell surface receptor of OPGL is mainly restricted to the osteoclasts. Hsu et al. (1999) Proc. Natl. Acad. Sci. USA 96: 3540-5. It is therefore an object of the invention to identify proteins and nucleic acids related to TNFs. Such proteins are believed to play a role in inflammatory and immune processes, suggesting their usefulness in treating autoimmune and inflammatory disorders.

A number of research groups have recently identified TNF family ligands with the same or substantially similar sequence, but they have not identified the associated receptor. The ligand has been variously named neutrokine α (WO 98/18921, published May 7, 1998), 63954 (WO 98/27114, published Jun. 25, 1998), TL5 (EP 869 180, published Oct. 7, 1998), NTN-2 (WO 98/55620 and WO 98/55621, published Dec. 10, 1998), TNRL1-alpha (WO 9911791, published Mar. 11, 1999), kay ligand (WO99/12964, published Mar. 18, 1999), and AGP-3 (U.S. Prov. App. Nos. 60/119,906, filed Feb. 12, 1999 and 60/166,271, filed Nov. 18, 1999, respectively). Each of these references is hereby incorporated by reference. A need exists in the art for a receptor binding to the ligands described in these references.

In unrelated research, Bram and von Bulow discovered a lymphocyte surface receptor named Transmembrane Activator and CAML Interactor (TACI) protein. See WO 98/39361, published Sep. 11, 1998, and von Bulow & Bram (1997), Science 278:138-140, which are hereby incorporated by reference. According to these references, TACI binds an intracellular cyclophilin ligand designated CAML, which modulates the calcium signaling pathway in lymphocytes.

### SUMMARY OF THE INVENTION

In accordance with the present invention, the inventors describe a receptor for neutrokine α, 63954, TL5, NTN-2, TNRL1-alpha, kay ligand, and AGP-3. The novel TNF ligand family member is herein called AGP-3 or TBAF (TNF family B cell Activation Factor ) and its receptor is herein named AGP-3 R-or TBAF R. Unlike other members of the family, the receptor for AGP-3 is primarily expressed in B cells, and its expression correlates to increases in the number of B cells and immunoglobulins produced.

The natural, preferred human ortholog of the receptor is here called hAGP-3Rand contains 273 amino acids. The AGP-3 R-protein is a type III transmembrane protein and has an N-terminal extracellular domain, a transmembrane domain, and a C-terminal intracellular domain.

The AGP-3-R-related proteins of the invention may be membrane-associated or in soluble form, recombinantly produced or isolated after natural production. Such proteins are useful for the treatment of autoimmune or inflammatory conditions, particularly B-cell related autoimmune or inflammatory conditions. AGP-3-R-related proteins comprising the extracellular domain of AGP-3 R, as well as antibodies to AGP-3R, are preferred for treatment of B-cell-related autoimmune or inflammatory conditions. A most preferred indication for AGP-3-R-related proteins and antibodies is lupus.

The present specification also describes nucleic acids encoding AGP-3 R-related proteins, vectors and host cells expressing the polypeptides, and methods for producing recombinant proteins. Antibodies or fragments thereof that specifically bind AGP-3 R-are also provided.

The subject proteins may be used in assays to identify cells and tissues that express AGP-3 R-or proteins related to AGP-3-R, and to identify new AGP-3 R-related proteins. Methods of identifying compounds that interact with AGP-3 R-proteins are also provided. Such compounds include nucleic acids, peptides, proteins, carbohydrates, lipids or small molecular weight organic molecules and may act either as agonists or antagonists of AGP-3 R-protein activity.

AGP-3 R related proteins are involved in B cell growth, survival, and activation, particularly in the lymph node, spleen, and Peyer's patches. AGP-3R agonists and antagonists (e.g., molecules incorporating the preferred regions of AGP-3 described below) thus modulate B cell response and may be used to treat diseases characterized by inflammatory processes or deregulated immune response, such as RA, GvHD, CD, lupus, and the like. Methods of use and pharmaceutical compositions comprising AGP-3 R-related proteins and AGP-3R agonists and antagonists are also encompassed by the invention.

In addition to therapeutic applications, AGP-3R related proteins may also be useful in production of hybridoma cells, which are derived from B cells. Thus, the present invention also concerns a method for modulating hybridoma cell antibody production, which comprises treating hybridoma cells with AGP-3R-related proteins.

### DESCRIPTION OF THE FIGURES

FIG. 1 shows the sequence of human AGP-3. Nucleic acid and amino acid sequences of human AGP-3 are indicated (SEQ ID NOS: 1 and 2, respectively). The predicted transmembrane region is underlined. Potential N-linked glycosylation sites are shown in boldface.

FIG. 2 shows the sequence of murine AGP-3. Nucleic acid and amino acid sequences of murine AGP-3 are indicated (SEQ ID NOS: 3 and 4, respectively). The predicted transmembrane region is underlined. Potential N-linked glycosylation sites are shown in boldface.

FIG. 3 shows an alignment of human and murine AGP-3, along with a consensus sequence (SEQ ID NO: 5). The predicted human and murine AGP-3 protein sequences were aligned by Pileup with gap creation penalty (12) and gap extension penalty (4) (Wisconsin GCG Package, Version 8.1, Genetics Computer Group Inc., Madison, Wis.). The consensus sequence was determined by Lineup (Wisconsin GCG Package, Version 8.1). The transmembrane regions from amino acid 47 to 72 in human AGP-3 and from amino acid 48 to 73 in murine AGP-3 are underlined. The N-terminal intracellular domain resides from amino acid 1 to 46 in human AGP-3 and from amino acid 1 to 47 in murine AGP-3. The C-terminal extracellular domain is localized from amino acid 73 to 285 in human AGP-3, and from amino acid 74 to 309. The human and murine AGP-3 share 68% amino acid identity overall. The C-terminus of AGP-3 is more conserved between human and mouse, with 87% identity over a 142-amino acid length. The putative conserved beta strands are indicated at the top, with the amino acids forming the putative strands underlined.

FIG. 4 shows human and murine AGP-3 mRNA tissue distribution. Human tissue northern blots (A) and murine tissue northern blots (B) were probed with ³²P-labeled human AGP-3 probe (A) or murine AGP-3 probe. The probed blots were exposed to Kodak film for 18 hours (A) or seven days (B).

FIG. 5 shows histology analysis of AGP-3 transgenic mouse spleen. The spleen sections from control mouse (A, C and E) and AGP-3 transgenic mouse (B, D, and F) were stained with hematoxylin and exosin (A and B), anti-mouse B220 (C and D) or anti-mouse CD3 (E and F). The spleen of the transgenic mouse was enlarged, mainly due to the increase of size and number of the follicles. The B cell staining areas in the spleen follicles in the transgenic mouse were enlarged. The T cell number was slightly diminished.

FIG. 6 shows histology analysis of AGP-3 transgenic mouse lymph nodes. The lymph node sections from control mouse (A, C and E) and AGP-3 transgenic mouse (B, D, and F) were stained with hematoxylin and exosin (A and B), anti-mouse B220 (C and D) or anti-mouse CD3 (E and F). The lymph node size of the transgenic mouse was enlarged. The B cell number was greatly increased in the transgenic mouse. Instead of restricted to marginal zones of the follicles as in the control mouse, the B cells also filled out the follicular area in the lymph nodes of the transgenic mouse. The T cell number was decreased in the transgenic mouse as compared to the control.

FIG. 7 shows histology analysis of AGP-3 transgenic mouse Peyer's patches. The Peyer's patches sections from control mouse (A, C and E) and AGP-3 transgenic mouse (B, D, and F) were stained with hematoxylin and exosin (A and B), anti-mouse B220 (C and D) or anti-mouse CD3 (E and F). The histologic and immunohistologic changes were similar to the changes in the lymph node of the transgenic mouse.

FIG. 8 shows FACS analysis of thymocytes, splenocytes and lymph node cells from AGP-3 transgenic mouse. Single-cell suspensions were prepared from spleen, lymph nodes and thymus from 10 AGP-3 transgenic mice and 5 control littermates. Cells were stained with FITC or PE-conjugated monoclonal antibodies against Thy-1.2, B220, CD1lb, Gr-1, CD4 or CD8. The B cell population increased by 100% in the transgenic mice as compared to the control mice. The T cell population decreased approximately 36%, with similar reductions in both CD4+ and CD8+ populations. Similar changes, though to a lesser degree, were observed in splenocytes. No differences in thymocyte staining were observed between the transgenic or control group.

FIG. 9 shows a sequence comparison of the C-terminal region of members of the TNF ligand family determined via Pileup (Wisconsin GCG Package, Version 8.1). Amino acid numbers are indicated on the left side. The putative conserved beta strands and loops are indicated at the top. The predicted N-glycosylation sites are indicated with asterisks. The top line shows the consensus sequence (SEQ ID NO: 6). The remaining lines show the sequence for the C-terminal region of the mammalian TNF-related protein identified (SEQ ID NOS: 7 to 24, 40).

FIG. 10 shows histology analysis of AGP-3 transgenic mice. Sections of spleen (A, B, C), lymph node (D, E, F) and Payer's patches (G, H, I) from control mice (left panel)and AGP-3 transgenic mice (right panel) were stained with hematoxylin and exosin (A, D, and G), anti-mouse B220 antibody (B, E, and H), or anti-mouse CD3 antibody (C, F, and I). Stained sections were analyzed under microscope at 10×.

FIG. 11 shows FACS analysis splenocytes, lymph node cells and thymocytes of AGP-3 transgenic mice. Single-cell suspensions were prepared form spleen, lymph nodes and thymus from 10 AGP-3 transgenic mice and 5 control littermates. Cells with stained with FITC or PE-conjugated monoclonal antibodies against thy-1.2, B220, CD11b, Gr-1, CD4 or CD8.

FIG. 12 shows elevation of serum immunoglobulin levels in AGP-3 transgenic mice. Control mice (n=5) and AGP-3 transgenic mice (n=5) were bled successively at 6, 7, 8, 9, 11 and 12 weeks of age. Serum IgM, IgG, IgA, and IgE levels were quantitated by ELISA. Values are expressed as Mean±SEM. All AGP-3 immunoglobulin levels were significantly increased (T-test; P<0.05) compared to control groups.

FIG. 13 shows kidney immunoglobulin deposits in AGP-3 transgenic mice. Kidney sections of 5 month control littermate (A, B, C), 5 month old AGP-3 mice (D, E, F), and 8 month old AGP-3 mice (G, H, I) were stained hematoxylin and exosin (A, D, and G), anti-mouse IgM (B, E, and H), anti-mouse IgG (C, F, and I), and Trichrome (G insert) Stained sections were analyzed under microscope at 60×.

FIG. 14 shows that AGP-3 stimulates B cell survival and proliferation.

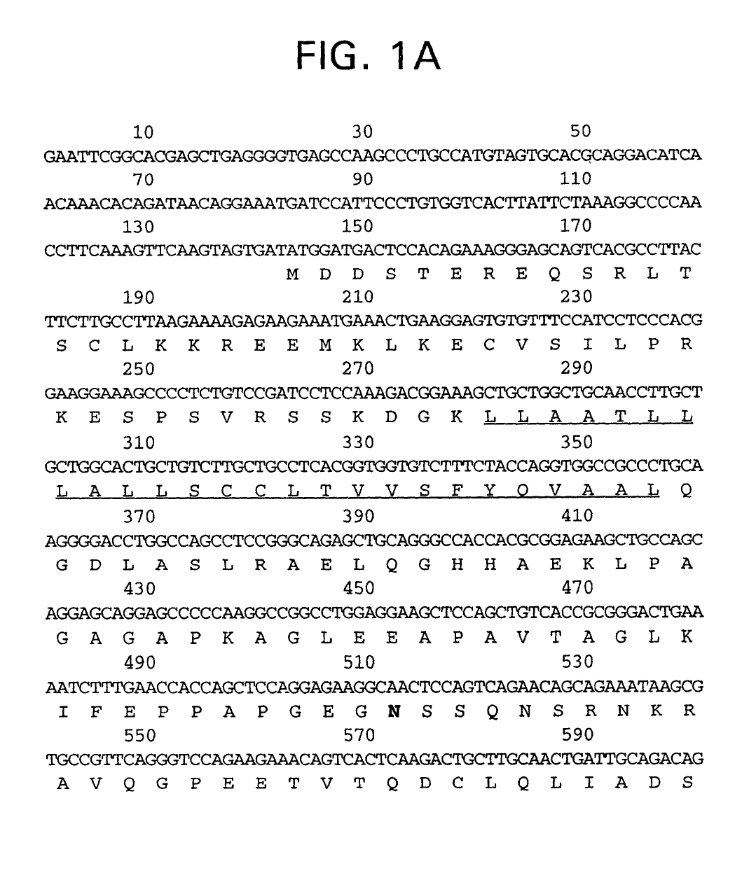

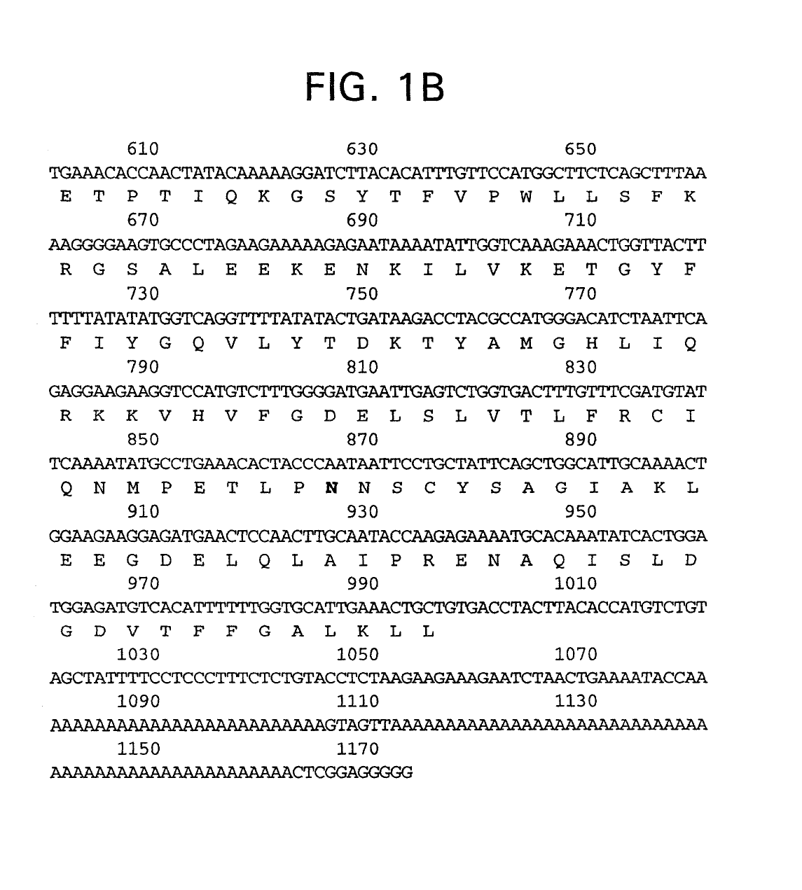

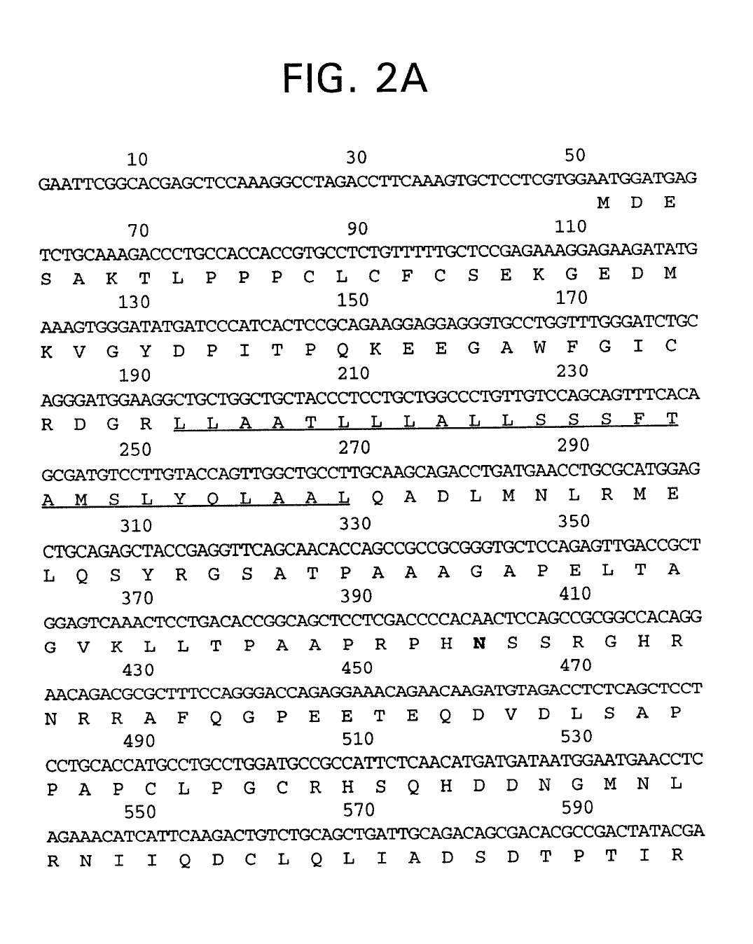

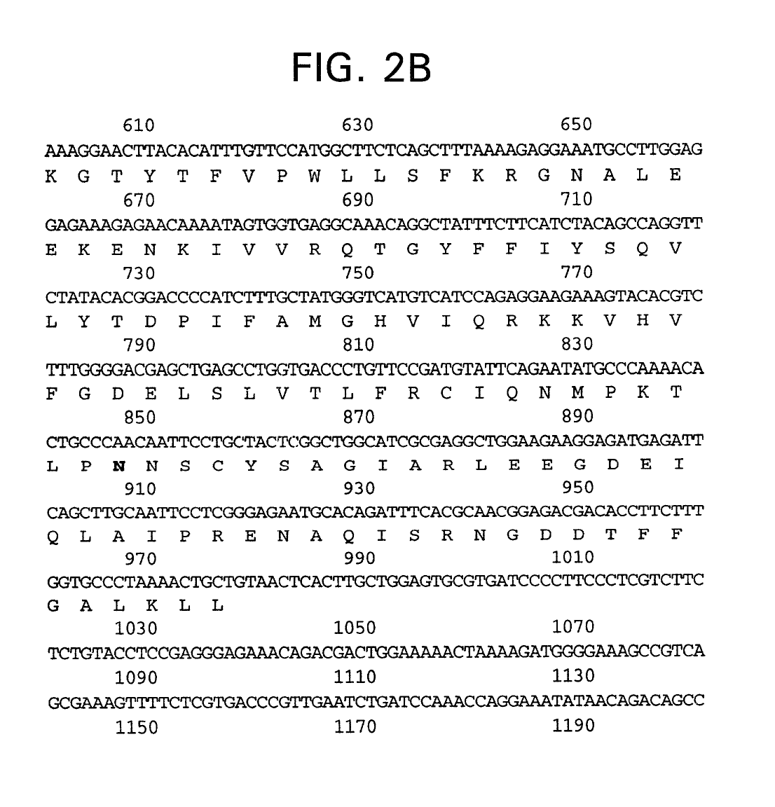

A. Increased B cell viability in AGP-3 transgenic mice. B cells were isolated from spleens of 3 month old AGP-3 transgenic mice (n−3) and control littermates (n=3). A total of 2.5×10⁵ B cells was aliquoted per well in a 96-well round bottom plate and incubated for 9 days. At the indicated days, cells were incubated with 5·g/ml Propidium Iodide and subject to FACS analysis for positive staining cells. Values are expressed as Mean±SEM.

B. AGP-3 stimulates B cell proliferation. Purified B cells (10⁵) from B6 mice were cultured in triplicates in 96 well plate with indicated amount of AGP-3 at the absence (upper panel) or presence of 2·g/ml anti-IgM antibody (lower panel) for a period of 4 days. Proliferation was measured by radioactive ³(H) thymidine uptake in last 18 hours of pulse. Data shown represent mean±standard deviation of triplicate wells.

FIG. 15 shows identification of AGP-3 receptor source. Approximately 10⁶ cells of each type were exposed to 1 μg/ml Flag-AGP-3 protein in the absence or presence of 10 μg/ml AGP-3 protein as specific competitor. Cells were subsequently incubated with 20 μg/ml anti-FLAG M2 monoclonal antibody, and then with 20 μg/ml FITC-conjugated goat anti-mouse IgG. Cells were finally analyzed by fluorescence activated cell sorting (FACS) using a Becton Dickinson FACscan.

FIG. 16 shows alignment of AGP-3 binding clones RAJI-13B4 and 13H11. The cDNA insert sequences from two positive binding clones 13B4 and 13H11 were aligned by GAP with gap creation penalty (12) and gap extension penalty (4) (Wisconsin GCG Package, Version 8.1, Genetics Computer Group Inc., Madison, Wis.). Two positive clones encode the same gene, with extra 7 bp at the N-terminus of clone 13H11.

FIG. 17 shows the nucleic acid and amino acid sequences (SEQ ID NOS: 41 and 42) of human AGP-3 receptor.

FIG. 18 shows the protein sequence of human AGP-3 receptor. The extracellular domain (SEQ ID NO: 43) includes the N-terminal domain (top line shown in FIG. 18, SEQ ID NO: 44) through two cysteine-rich repeats (labeled I and II, SEQ ID NOS: 45 and 46) to the end of the “stalk” region (SEQ ID NO: 47). The transmembrane domain (labeled TM, SEQ ID NO: 48) is underlined, and the intracellular domain (labeled IC, SEQ ID NO: 49) is also indicated.

FIG. 19. Alignment of extracellular domains of human AGP-3 receptor and TNFR1. Extracellular domain of human AGP-3 receptor and TNFR1 were aligned by GAP with gap creation penalty (12) and gap extension penalty (4) (Wisconsin GCG Package, Version 8.1, Genetics Computer Group Inc., Madison, Wis.).

FIG. 20. Northern analysis of human AGP-3 receptor. Human tissue northern blots were probed with ³²P-labeled human AGP-3 receptor probe. The probed blots were exposed to Kodak film for 18 hours.

FIG. 21. Extracellular domain of AGP-3R binds to AGP-3.

A. Western analysis of AGP-3R-Fc fusion protein. 293 cells were transfected with control vector or AGP-3R-Fc/pCEP4 expression vector which directs synthesis extracellular domain of AGP-3R fused human IgG Fc at the C-terminus. After 24 hour transfection, medium and cell lysates were subject to western analysis with anti-Fc antibody. The AGP-3R-Fc fusion protein was detected only in the transfected cell lysates, not in the medium. This supports that AGP-3 receptor extracellular domain lacks a N-terminal signal peptide.

B. AGP-3R-Fc fusion protein binds AGP-3. Cell lysates that contain AGP-3R-Fc fusion protein generated as described above were incubated with FLAG-AGP-3 protein and protein A beads for 1 hr at 4° C. The protein A beads were washed with E1A buffer for 6 times. The precipitates were fractionated by SDS-PAGE and subject to western blot analysis by anti-FLAG antibody. FLAG-AGP-3 was co-precipitated by AGP-3R-Fc cell lysates.

### AGP-3DETAILED DESCRIPTION OF THE INVENTION

Definition of Terms

The following definitions apply to the terms used throughout this specification, unless otherwise limited in specific instances.

The term “AGP-3 related protein” refers to natural and recombinant proteins comprising the following sequence:

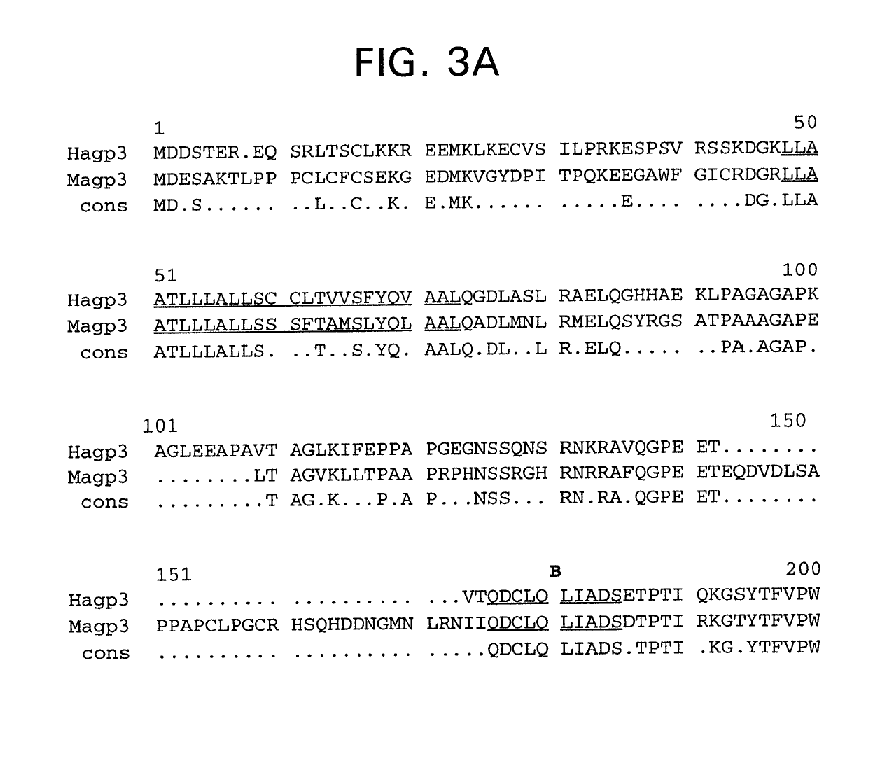

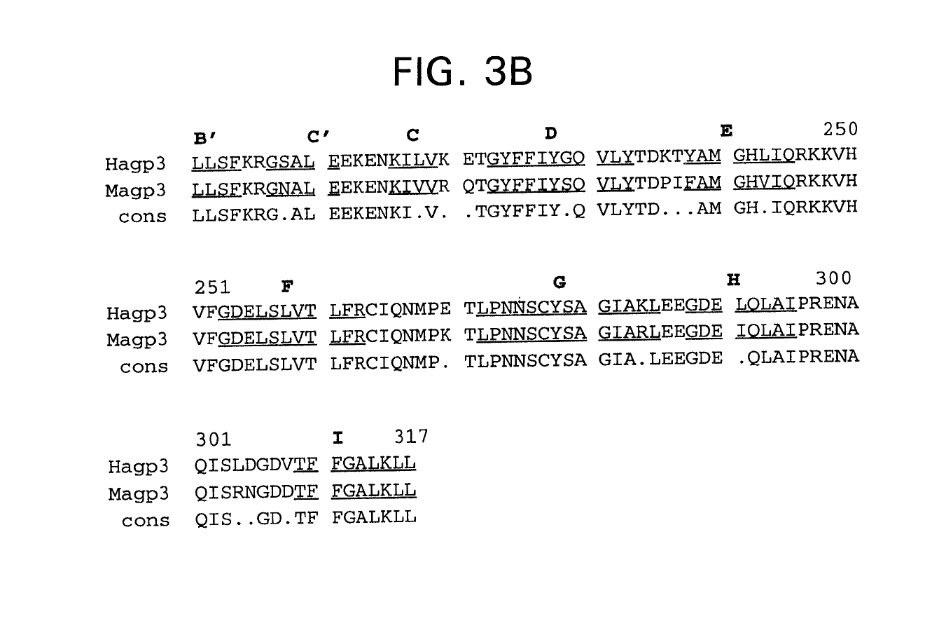

QDCLQLIADSXTPTIXKGXYTFVPWLLSF (SEQ ID NO: 25)

wherein “X” may be any naturally occurring amino acid residue. This sequence is a consensus of the B and B′ β-sheets and B/B′ loop of hAGP-3 and mAGP-3 (see FIG. 3), which is believed to be the specific receptor binding site. Preferred AGP-3-related proteins comprise both the B/B′ consensus and the E/F consensus:

AMGHXIQRKKVHVFGDELSLVTLFR (SEQ ID NO: 26)

The E/F region is also believed to be involved in receptor binding. More preferred proteins are those comprising the consensus of the B-I region:

QDCLQLIADS XTPTIXKGXY TFVPWLLSFK RGXALEEKEN KIXVXXTGYF (SEQ ID NO: 27)
FIYXQVLYTD XXXAMGHXIQ RKKVHVFGDE LSLVTLFRCI QNMPXTLPNN
SCYSAGIAXL EEGDEXQLAI PRENAQISXX GDXTFFGALK LL

“AGP-3-related activity” means that a natural or recombinant protein (including antibodies), analog, derivative or fragment that (a) is capable of interacting with an AGP-3-related protein or (b) has the same binding site on an AGP-3R-related protein as an AGP-3-related protein, and thereby is capable of modulating B cell growth, survival, or activation. Of particular interest is such AGP-3-related activity in MLN, spleen, and Peyer's patches. The inventors contemplate that some molecules of interest may have activity antagonistic to native AGP-3 activity; for example, a derivative or analog may retain AGP-3 binding activity but will not activate the AGP-3 receptor. All such activity (agonism and antagonism of AGP-3) falls within the meaning of “AGP-3 related activity.” Such activity can be determined, for example, by such assays as described in “Biological activity of AGP-3” in the Materials & Methods hereinafter, which may be modified as needed by many methods known to persons having ordinary skill in the art.

The term “AGP-3R related protein” refers to proteins comprising the cystein-rich repeats (SEQ ID NOS: 45 and 46) of the extracellular domain (SEQ ID NO: 43) of AGP-3-R. Such proteins having at least about 80% identity with the extracellular domain are preferred, with those having 90% or 95% identity or greater more preferred. Most preferred proteins comprise the sequence or sequences of the amino acids that interact with the B/B′ and/or E/F regions of AGP-3, or more generally with the B-I region of AGP-3. Such sequences can be included in naturally occurring proteins, truncated naturally occurring proteins, or recombinant and synthetic proteins. Recombinant and synthetic AGP-3R-related proteins may be formed by fusion of the AGP-3R-derived fragment with unrelated molecules or molecular domains (e.g., Fc regions), domain swapping with other TNF receptor family members, antibody grafting (e.g., substituting an AGP-3R fragment sequence for an antibody CDR or variable domain), or other modifications. Such proteins are discussed further hereinbelow. The proteins may also be modified by linkage to a carbohydrate (e.g., dextran) or a water-soluble polymer (e.g., PEG). The proteins within this definition may also include substitution with amino acids serving as sites for attachment of non-protein groups (e.g., glycosylation sites). All such proteins are encompassed by the terms “AGP-3R related protein.”

An “analog” of an AGP-3R protein (e.g., hAGP-3R) is a polypeptide within the definition of “AGP-3R-related protein” that has a substitution or addition of one or more amino acids. Such an AGP-3R-related protein should maintain the property of eliciting B cell growth, survival, or activation. Such analogs will have substitutions or additions at any place along the polypeptide. Preferred analogs include those of soluble AGP-3R-related proteins. Fragments or analogs may be naturally occurring, such as a polypeptide product of an allelic variant or a mRNA splice variant, or they may be constructed using techniques available to one skilled in the art for manipulating and synthesizing nucleic acids. The polypeptides may or may not have an amino terminal methionine residue.

A “derivative” of an AGP-3R-protein is a polypeptide within the definition of “AGP-3R-related protein” that has undergone post-translational modifications. Such modifications include, for example, addition of N-linked or O-linked carbohydrate chains, processing of N-terminal or C-terminal ends, attachment of chemical moieties to the amino acid backbone, chemical modifications of N-linked or O-linked carbohydrate chains, and addition of an N-terminal methionine residue due to prokaryotic host cell expression. In particular, chemically modified derivatives of AGP-3R-related protein that provide additional advantages such as increased stability, longer circulating time, or decreased immunogenicity are contemplated. Of particular use is modification with water soluble polymers, such as polyethylene glycol and derivatives thereof (see for example U.S. Pat. No. 4,179,337). The chemical moieties for derivitization may be selected from water-soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties. Polypeptides may also be modified at pre-determined positions in the polypeptide, such as at the amino terminus, or at a selected lysine or arginine residue within the polypeptide. Other chemical modifications provided include a detectable label, such as an enzymatic, fluorescent, isotopic or affinity label to allow for detection and isolation of the protein.

The term “protein” refers to polypeptides regardless of length or origin, comprising molecules that are recombinantly produced or naturally occurring, full length or truncated, having a natural sequence or mutated sequence, with or without post-translational modification, whether produced in mammalian cells, bacterial cells, or any other expression system.

Ligand for the Receptor

The natural, preferred human ortholog of the associated ligand (hAGP-3) contains 285 amino acids; the mouse ortholog (mAGP-3), contains 309 amino acids. The AGP-3 protein is a type II transmembrane protein and has an amino terminal cytoplasmic domain, a transmembrane domain, and a carboxy terminal extracellular domain. TNF-related proteins of the invention may be membrane-associated or in soluble form, recombinantly produced or isolated after natural production. The present specification demonstrates that AGP-3 is a potent B cell stimulatory factor. Interestingly, the AGP-3 transgenic mice also developed autoantibodies and kidney immune complex deposits, a phenotype resembling lupus patients and lupus prone mice.

AGP-3-related protein primarily acts on B cells. An EST bearing a portion of the AGP-3 sequence was obtained from a human fetal liver spleen cDNA library. A labeled cDNA fragment was used to probe a human spleen cDNA phage library (see “Cloning of Human AGP-3” in Materials & Methods hereinafter). The cDNA encoding a human AGP-3 was isolated from this phage library. The human protein is a type II transmembrane protein, having a short N-terminal intracellular region that differed from other members of the TNF ligand family and a long C-terminal extracellular region that comprises most of the conserved region of the TNF ligand family.

An EST encoding a murine ortholog of AGP-3 was identified by BLAST search of Genebank using the human AGP-3 sequence. The corresponding cDNA clone was obtained from a mouse lymph node library and used to probe a mouse spleen cDNA phage library (see Materials & Methods hereinafter). The cDNA encoding a murine AGP-3 ortholog was isolated from this phage library.

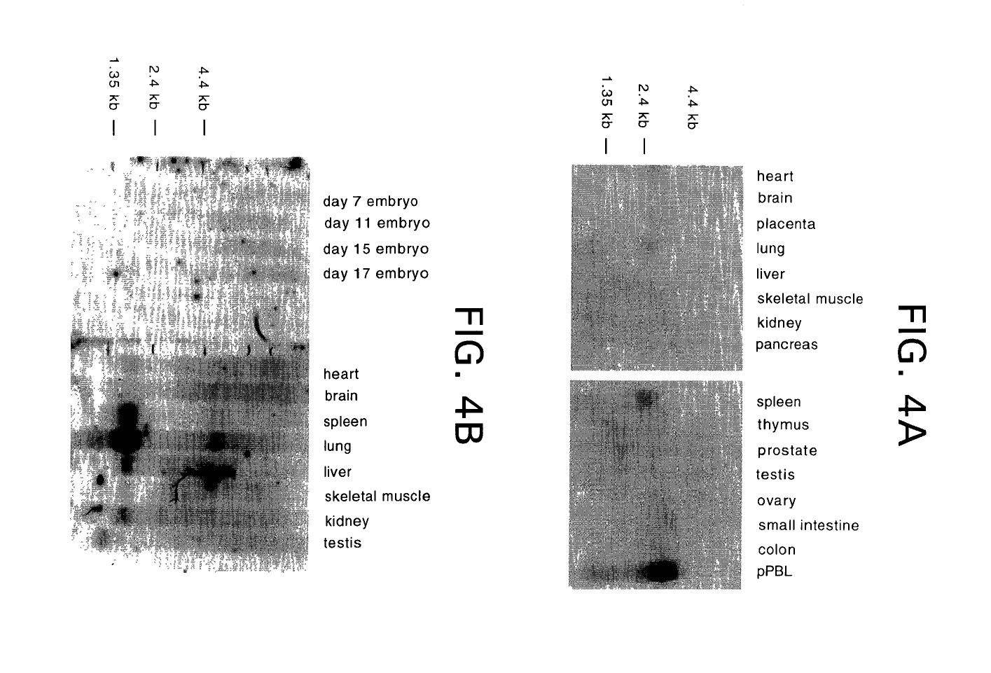

Northern blots were used to determine tissue distribution of transcription of AGP-3 (see “Cloning of Murine AGP-3” in Materials & Methods hereinafter). In murine tissue, AGP-3 mRNA was detected mainly in spleen, lung, liver, and kidney. In human tissue, AGP-3 mRNA was detected predominantly in peripheral blood leukocytes, with weaker transcription in spleen, lung, and small intestine (see FIGS. 4A and 4B).

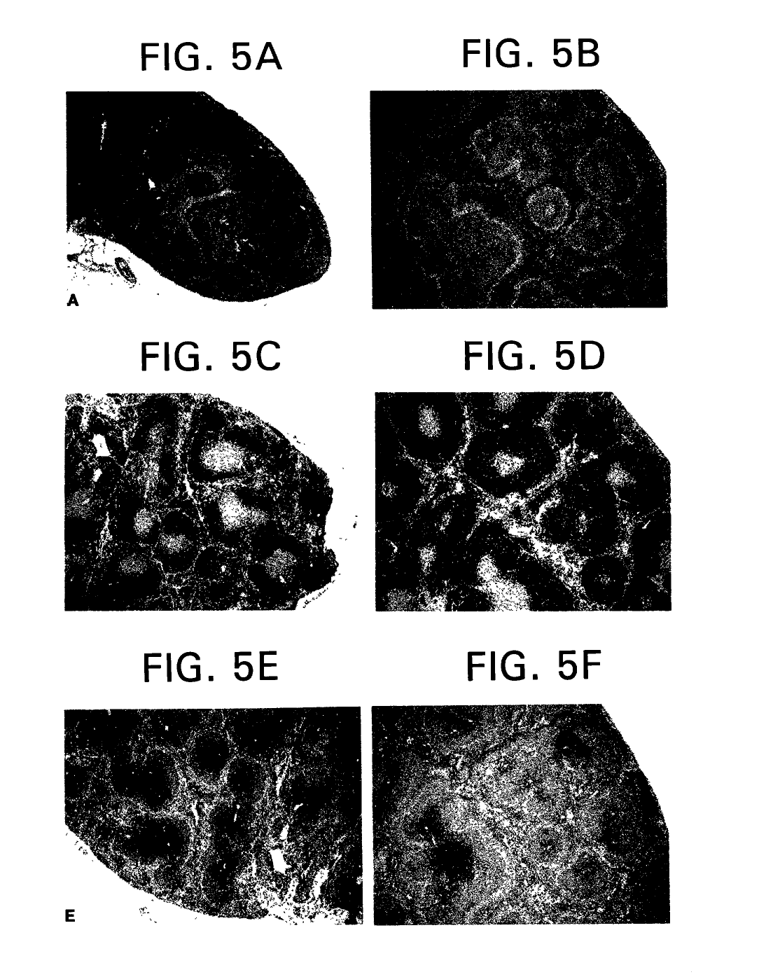

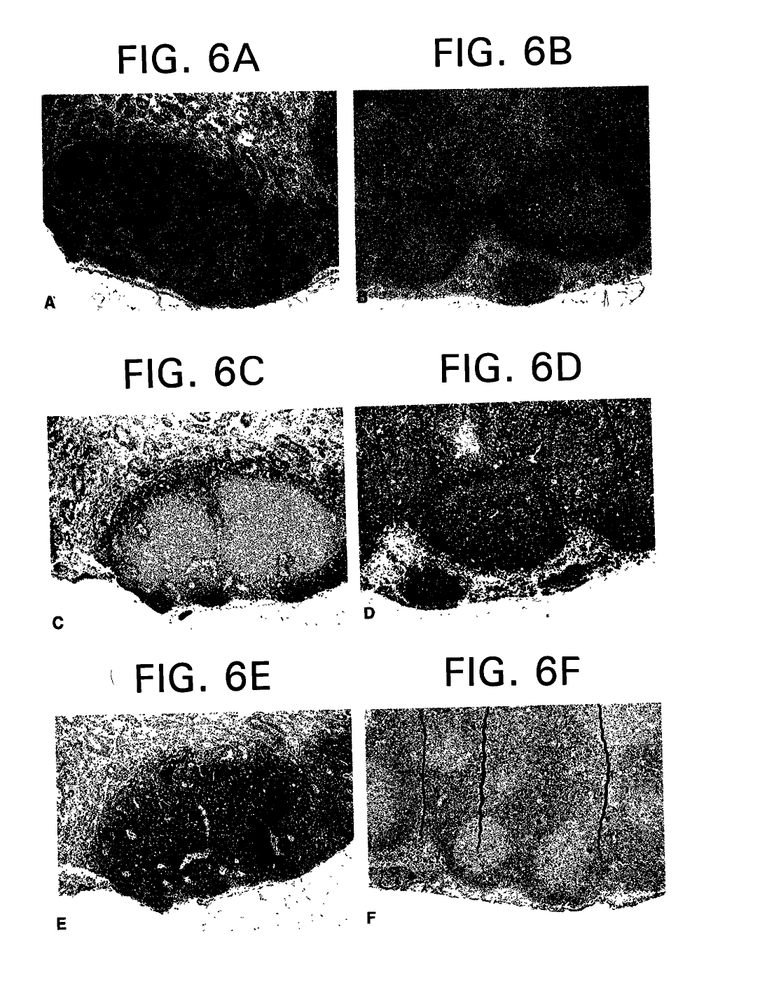

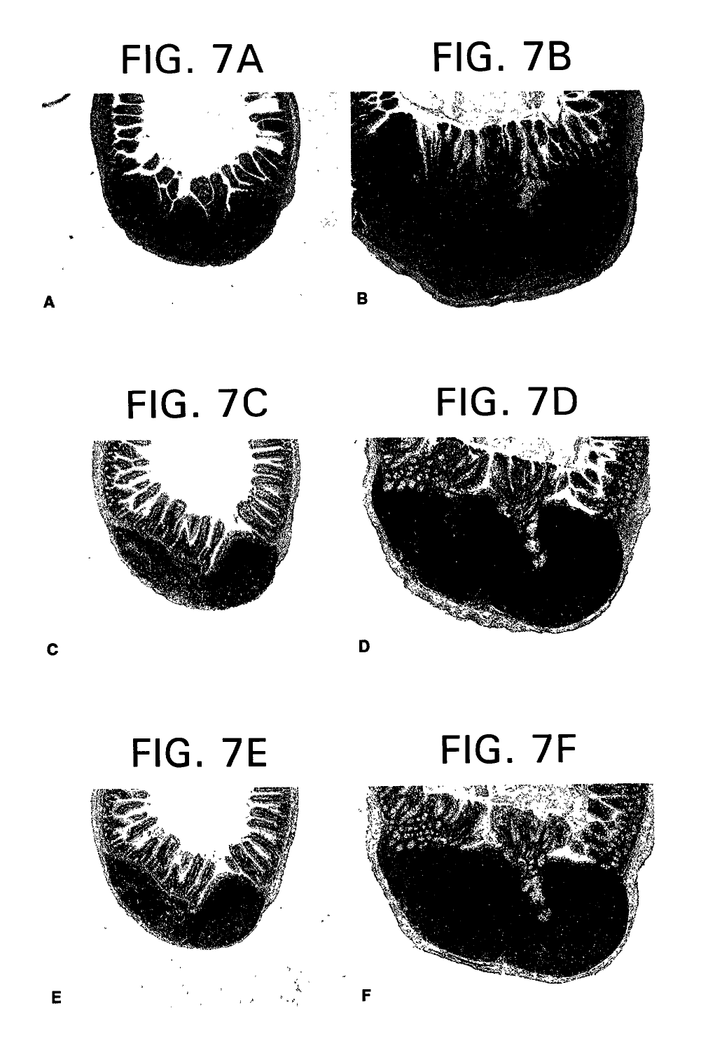

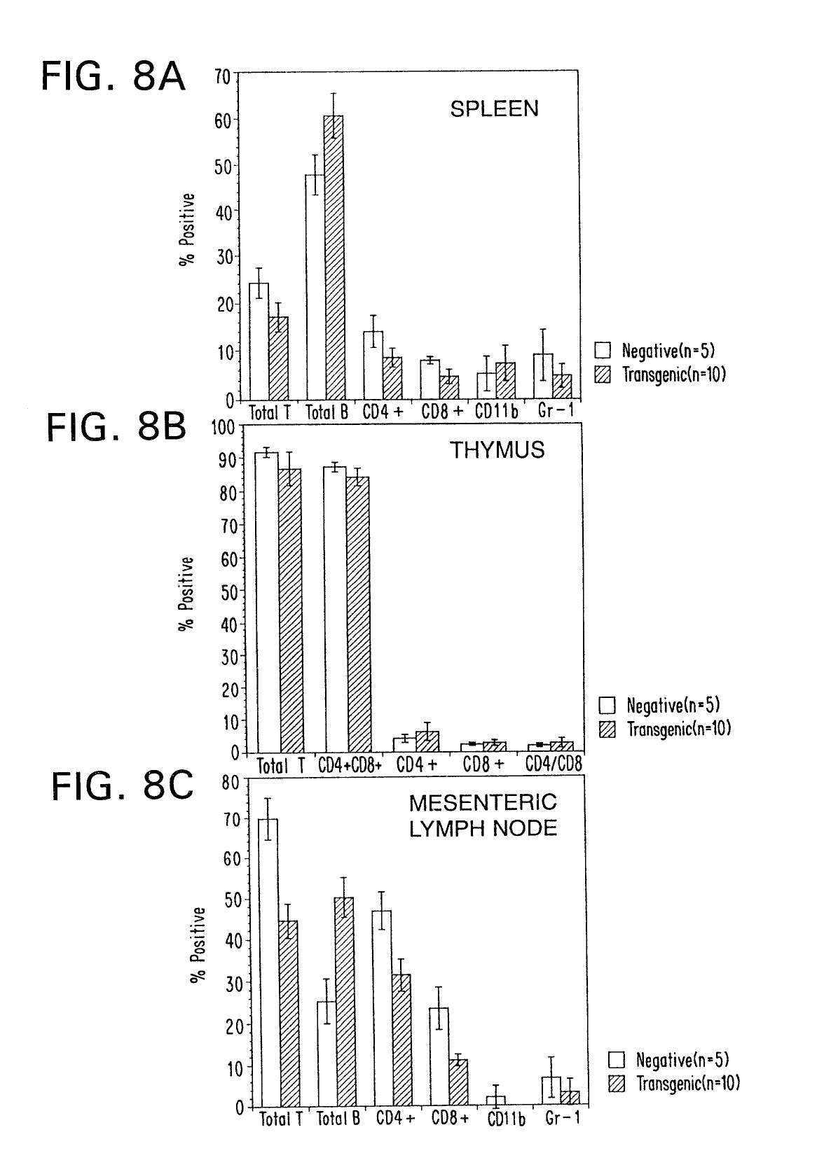

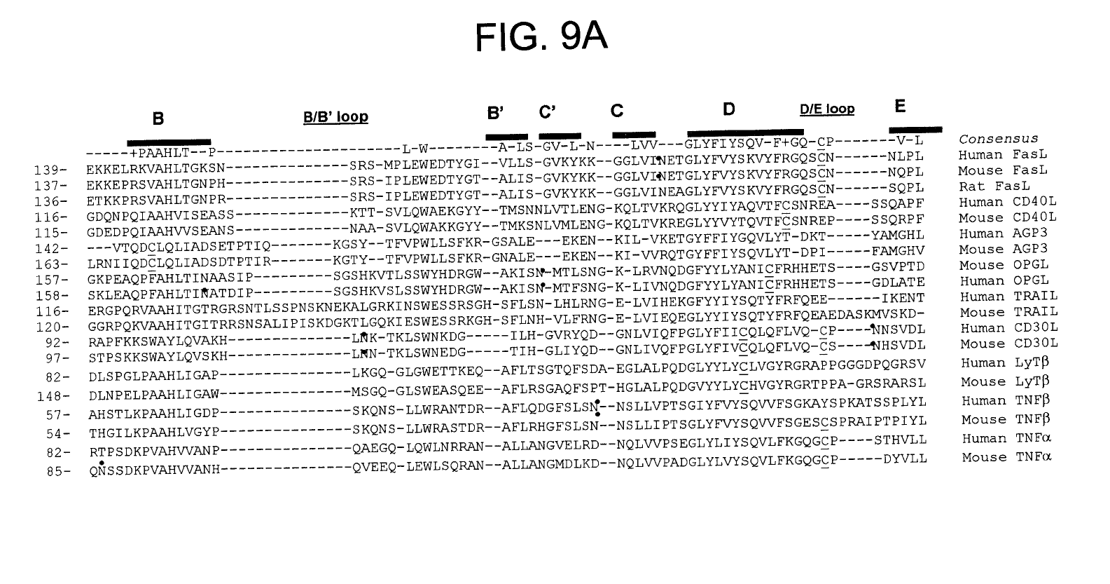

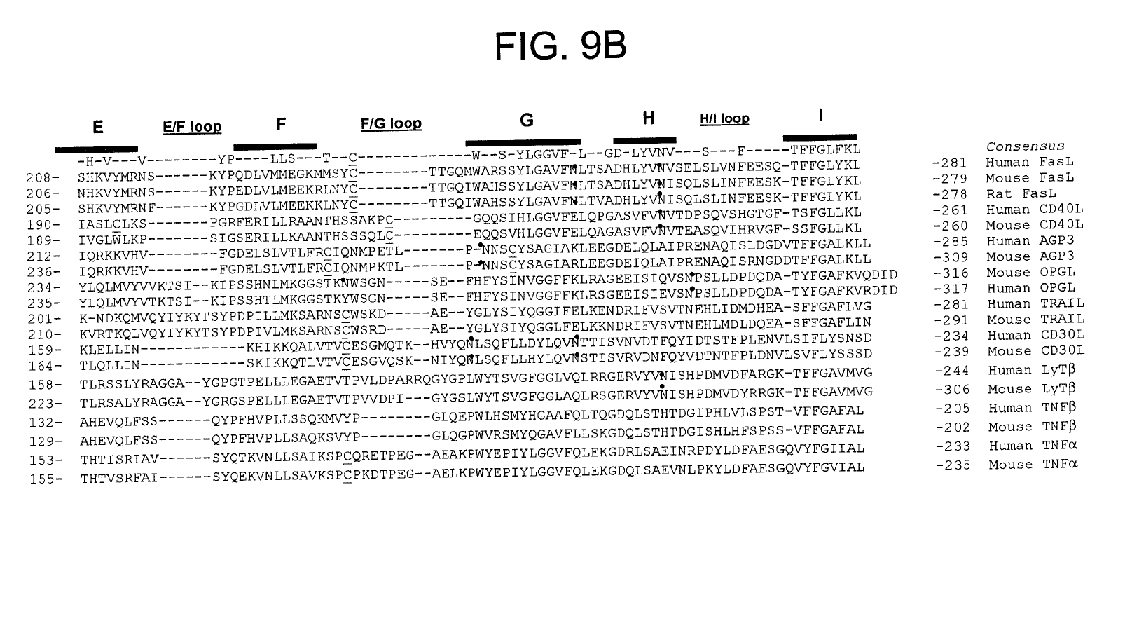

The murine ortholog of AGP-3 was overexpressed in transgenic mice (see “Overexpression of murine AGP-3 in transgenic mice” in Materials & Methods hereinafter). In these transgenic mice, serum globulin and total protein levels increased greatly over control littermates while the albumin level remained the same (see “Biological Activity of AGP-3” in Materials & Methods hereinafter). The mice also exhibited increases in the size and number of follicles in the spleen, lymph nodes, and Peyer's patches (FIGS. 5, 6, and 7). In their MLN, the mice exhibited 100% increases in the number of cells expressing CD45 receptor with concomitant decreases in cells expressing CD90, CD4, and CD8. These results correspond to an increase in the B cell population and a decrease in the T cell population in the MLN (FIGS. 6 and 8). Similar results were obtained in the spleen, but to a lesser extent (FIGS. 5 and 8).

Nucleic Acids

The invention provides for isolated nucleic acids encoding AGP-3 R-related proteins. As used herein, the term nucleic acid comprises cDNA, genomic DNA, wholly or partially synthetic DNA, and RNA. These nucleic acids may be prepared or isolated as described in the working examples hereinafter or by nucleic acid hybridization thereof.

Nucleic acid hybridization typically involves a multi-step process. A first hybridization step forms nucleic acid duplexes from single strands. A second hybridization step under more stringent conditions selectively retains nucleic acid duplexes having the desired homology. The conditions of the first hybridization step are generally not crucial, provided they are not of higher stringency than the second hybridization step. Generally, the second hybridization is carried out under conditions of high stringency, wherein “high stringency” conditions refers to conditions of temperature and salt that are about 12-20° C. below the melting temperature (Tₘ) of a perfect hybrid of part or all of the complementary strand corresponding to the AGP-3R extracellular domain shown in FIG. 17. In one embodiment, “high stringency” conditions refer to conditions of about 65° C. and not more than about 1 M Na⁺. It is understood that salt concentration, temperature and/or length of incubation may be varied in either the first or second hybridization steps such that one obtains the hybridizing nucleic acid molecules according to the invention. Conditions for hybridization of nucleic acids and calculations of Tₘ for nucleic acid hybrids are described in Sambrook et al. (1989), Molecular Cloning: A Laboratory Manual Cold Spring Harbor Laboratory Press, New York.

The nucleic acids of the invention may hybridize to part or all of the polypeptide coding regions of AGP-3R related proteins (e.g., as shown in FIG. 17) and therefore may be truncations or extensions of the nucleic acid sequences shown therein. Truncated or extended nucleic acids are encompassed by the invention provided that the encoded proteins retain AGP-3 related activity. In one embodiment, the nucleic acid will encode a polypeptide of at least about 10 amino acids. In another embodiment, the nucleic acid will encode a polypeptide of at least about 20 amino acids. In yet another embodiment, the nucleic acid will encode a polypeptide of at least about 50 amino acids. The hybridizing nucleic acids may also include noncoding sequences located 5′ and/or 3′ to the coding regions for the AGP-3R related protein. Noncoding sequences include regulatory regions involved in expression of AGP-3R-related protein, such as promoters, enhancer regions, translational initiation sites, transcription termination sites and the like.

In preferred embodiments, the nucleic acids of the invention encode human AGP-3-R. Most preferred are the nucleic acids encoding the extracellular domain. For molecules encoding AGP-3R comprising the transmembrane domain, substitutions that replace hydrophobic amino acid residues in this region with neutral or hydrophilic amino acid residues would be expected to disrupt membrane association and result in soluble AGP-3R-related protein. In addition, deletions of part or all the transmembrane region would also be expected to produce soluble forms of AGP-3R-related protein.

Nucleic acid sequences of the invention may also be used for the detection of sequences encoding AGP-3R-related protein in biological samples. In particular, the sequences may be used to screen cDNA and genomic libraries for AGP-3R-related protein sequences, especially those from other species. The nucleic acids are also useful for modulating levels of AGP-3 R-related protein by anti-sense technology or in vivo gene expression. Development of transgenic animals expressing AGP-3R-related protein are useful for production of the polypeptides and for the study of in vivo biological activity.

Vectors and Host Cells

The nucleic acids of the invention will be linked with DNA sequences so as to express biologically active AGP-3 R-related protein. Sequences required for expression are known to those skilled in the art and include promoters and enhancer sequences for initiation of RNA synthesis, transcription termination sites, ribosome binding sites for the initiation of protein synthesis, and leader sequences for secretion. Sequences directing expression and secretion of AGP-3 R-related protein may be homologous, i.e., the sequences are identical or similar to those sequences in the genome involved in AGP-3 R-related protein expression and secretion, or they may be heterologous. A variety of plasmid vectors are available for expressing AGP-3R-related protein in host cells; see, for example, Methods in Enzymology v. 185, Goeddel, D. V. ed., Academic Press (1990). For expression in mammalian host cells, a preferred embodiment is plasmid pDSRα described in PCT Application No. 90/14363. For expression in bacterial host cells, preferred embodiments include plasmids harboring the lux promoter (see co-owned and co-pending U.S. Ser. No. 08/577,778, filed Dec. 22, 1995). In addition, vectors are available for the tissue-specific expression of AGP-3 R-related protein in transgenic animals. Gene transfer vectors derived from retrovirus (RV), adenovirus (AdV), and adeno-associated virus (AAV) may also be used for the expression of AGP-3R-related protein in human cells for in vivo therapy (see PCT Application No. 86/00922).

Prokaryotic and eukaryotic host cells expressing AGP-3 R-related protein are also provided by the invention. Host cells include bacterial, yeast, plant, insect or mammalian cells. AGP-3 R-related protein may also be produced in transgenic animals, such as mice or goats. Plasmids and vectors containing the nucleic acids of the invention are introduced into appropriate host cells using transfection or transformation techniques known to one skilled in the art. Host cells may contain DNA sequences encoding AGP-3 R-related protein as shown in FIG. 17 or a portion thereof, such as the extracellular domain or the cytoplasmic domain. Nucleic acids encoding AGP-3 R-related proteins may be modified by substitution of codons that allow for optimal expression in a given host. At least some of the codons may be so-called preference codons that do not alter the amino acid sequence and are frequently found in genes that are highly expressed. However, it is understood that codon alterations to optimize expression are not restricted to the introduction of preference codons. Examples of preferred mammalian host cells for AGP-3 R-related protein expression include, but are not limited to COS, CHOd-, 293 and 3T3 cells. A preferred bacterial host cell is Escherichia coli.

Polypeptides

The invention also provides AGP-3 R-related protein as the products of prokaryotic or eukaryotic expression of an exogenous DNA sequences. Exogenous DNA sequences include cDNA, genomic DNA and synthetic DNA sequences. AGP-3 R-related proteins may be the products of bacterial, yeast, plant, insect or mammalian cells expression, or from cell-free translation systems. AGP-3 R-related proteins produced in bacterial cells will have N-terminal methionine residues. The invention also provides for a process of producing AGP-3 R-related proteins comprising growing prokaryotic or eukaryotic host cells transformed or transfected with nucleic acids encoding them and isolating polypeptide expression products of the nucleic acids.

Polypeptides that are mammalian proteins or are fragments, analogs or derivatives thereof are encompassed by the invention. In preferred embodiments, the AGP-3R-related protein is human AGP-3 R. A fragment of AGP-3R-related protein refers to a polypeptide having a deletion of one or more amino acids such that the resulting polypeptide retains AGP-3 related activity; for example, the polypeptide has at least the property of antagonizing B cell growth, survival, or activation, especially in mesenteric lymph nodes. Said fragments will have deletions originating from the amino terminal end, the carboxy terminal end, or internal regions of the polypeptide. Fragments of AGP-3 R-related proteins are at least about ten amino acids, at least about 20 amino acids, or at least about 50 amino acids in length. In preferred embodiments, AGP-3 R-related proteins will have a deletion of one or more amino acids from the transmembrane region (see FIG. 17), or, alternatively, one or more amino acids from the amino-terminus up to and/or including the transmembrane region.

The polypeptides of the invention are isolated and purified from tissues and cell lines that express AGP-3 R-related protein, either extracted from lysates or from conditioned growth medium, and from transformed host cells expressing AGP-3 R-related protein. Human AGP-3 R-related protein, or nucleic acids encoding same, may be isolated from human lymph node or fetal liver tissue. Isolated AGP-3 R-related protein is free from association with human proteins and other cell constituents.

A method for purification of such proteins from natural sources (e.g. tissues and cell lines that normally express an AGP-3R related protein) and from transfected host cells is also encompassed by the invention. The purification process may employ one or more standard protein purification steps in an appropriate order to obtain purified protein. The chromatography steps can include ion exchange, gel filtration, hydrophobic interaction, reverse phase, chromatofocusing, affinity chromatography employing an anti-AGP-3 R-related protein antibody or biotin-streptavidin affinity complex and the like.

Fusion Proteins and Derivatives

The invention further comprises AGP-3 R-related protein chimeras, as well as such proteins derivatized by linkage to such molecules as PEG or dextran. Such proteins comprise part or all of an AGP-3 R-related protein amino acid sequence fused to a heterologous amino acid sequence. The heterologous sequence may be any sequence that allows the resulting fusion protein to retain AGP-3 R-related activity. In preferred embodiments, a heterologous sequence is fused to a portion of an AGP-3 R-related protein's sequence that interacts with an AGP-3 related protein's B/B′ region (SEQ ID NO: 25) and/or the E/F region (SEQ ID NO: 26) or with the more complete B-I region (SEQ ID NO: 27). Such heterologous sequences include cytoplasmic domains that allow for alternative intracellular signaling events, sequences that promote oligomerization (e.g., the Fc region of IgG), enzyme sequences that provide a label for the polypeptide, and sequences that provide affinity probes (e.g., an antigen-antibody recognition site).

Preferred molecules in accordance with this invention are Fc-linked AGP-3 R-related proteins. Useful modifications of protein therapeutic agents by fusion with the “Fc” domain of an antibody are discussed in detail in a patent application entitled, “Modified Peptides as Therapeutic Agents,” U.S. Ser. No. 09/428,082, PCT appl. no. WO 99/25044, which is hereby incorporated by reference in its entirety. That patent application discusses linkage to a “vehicle” such as PEG, dextran, or an Fc region.

In the compositions of matter prepared in accordance with this invention, the AGP-3 R-related protein may be attached to a vehicle through the protein's N-terminus or C-terminus. Thus, the vehicle-protein molecules of this invention may be described by the following formula I:

(X¹)ₐ—F¹—(X²)_{b} I

wherein:

F¹ is a vehicle (preferably an Fc domain);

X¹ and X² are each independently selected from —(L¹)_{c}—P¹, —(L¹)_{c}—P¹—(L²)_{d}—P²,—(L¹)_{c}—P¹—(L²)_{d}—P²—(L³)ₑ—P³, and —(L¹)_{c}—P¹—(L²)_{d}—P²—(L³)ₑ—P³—(L⁴)_{f}—P⁴

P¹, P², P³, and P⁴ are each independently sequences of an AGP-3 R-related protein (e.g., a fragment of hAGP-3R that is capable of binding to AGP-3) and are preferably selected from SEQ ID NO: 38, 39, 40, and 41;

L¹, L², L³, and L⁴ are each independently linkers; and

a, b, c, d, e, and f are each independently 0 or 1, provided that at least one of a and b is 1.

Thus, compound I comprises preferred compounds of the formulae

X¹—F¹ II

and multimers thereof wherein F¹ is an Fc domain and is attached at the C-terminus of X¹;

F¹—X² III

and multimers thereof wherein F¹ is an Fc domain and is attached at the N-terminus of X²;

F¹—(L¹)_{c}—P¹ IV

and multimers thereof wherein F¹ is an Fc domain and is attached at the N-terminus of —(L¹)_{c}—P¹; and

F¹—(L¹)_{c}—P¹—(L²)_{d}—P² V

and multimers thereof wherein F¹ is an Fc domain and is attached at the N-terminus of —L¹—P¹—L²—P².

Antibodies

Uses for antibodies specifically binding the polypeptides of the invention are also encompassed by the invention. The antibodies may be generated by immunization with full-length AGP-3R related protein, or fragments thereof. Preferred antibodies bind to the portions of AGP-3R that interact with the B/B′ and/or E/F regions of AGP-3 or more generally with the B-I region. Such antibodies may be generated by immunization with polypeptides comprising those portions of AGP-3R. The term “antibodies” also refers to molecules having Fv, Fc and other structural domains usually associated with antibodies but that may be generated by other techniques (e.g., phage display antibody generation). The antibodies of the invention may be polyclonal or monoclonal, or may be recombinant antibodies, such as chimeric antibodies wherein the murine constant regions on light and heavy chains are replaced by human sequences, or CDR-grafted antibodies wherein only the complementarity determining regions are of murine origin. Antibodies of the invention may also be fully human antibodies prepared, for example, by immunization of transgenic animals capable of producing human antibodies (see, for example, PCT Application No. WO93/12227). Regardless of the means by which they are generated, antibodies in accordance with this invention may be produced by recombinant means (e.g., transfection of CHO cells with vectors comprising antibody sequence).

The antibodies are useful for detecting AGP-3R-related protein in biological samples, thereby allowing the identification of cells or tissues that produce such proteins. In addition, antibodies that bind to AGP-3R related proteins and block interaction with other binding compounds (i.e., “antagonist antibodies”) have therapeutic use in modulating B cell growth, activation, and/or proliferation. On the other hand, antibodies that bind to AGP-3R and activate the receptor as would AGP-3 (“agonist antibodies”) have therapeutic use in conditions in which the patient would benefit from B cell growth, activation or proliferation (e.g., in patients immunocompromised due to chemotherapy or acquired immune deficiency syndrome). Antibodies can be tested for binding to AGP-3R related protein and examined for their effect on AGP-3-mediated B cell growth, survival, or activation associated with the disease or condition (see “Biological activity of AGP-3” in Materials & Methods hereinafter).

Compositions

The invention also provides for pharmaceutical compositions comprising a therapeutically effective amount of the AGP-3 related protein or AGP-3 R-related protein of the invention together with a pharmaceutically acceptable diluent, carrier, solubilizer, emulsifier, preservative and/or adjuvant. The invention also provides for pharmaceutical compositions comprising a therapeutically effective amount of an AGP-3 related or AGP-3 R-related protein agonist or antagonist. The term “therapeutically effective amount” means an amount that provides a therapeutic effect for a specified condition and route of administration. The composition may be in a liquid or lyophilized form and comprises one or more of the following:

a diluent (e.g., Tris, acetate or phosphate buffers) having various pH values and ionic strengths;

a solubilizer (e.g., Tween or Polysorbate);

carriers (e.g., human serum albumin or gelatin);

preservatives (e.g., thimerosal or benzyl alcohol); and

antioxidants (e.g., ascorbic acid or sodium metabisulfite).

Selection of a particular composition will depend upon a number of factors, including the condition being treated, the route of administration and the pharmacokinetic parameters desired. A more extensive survey of component suitable for pharmaceutical compositions is found in Remington's Pharmaceutical Sciences (1980), 18th ed. (A. R. Gennaro, ed.) Mack, Easton, Pa.

In a preferred embodiment, compositions comprising soluble AGP-3 R-related proteins are provided. Also encompassed are compositions comprising soluble AGP-3R-related protein modified with water-soluble polymers to increase solubility, stability, plasma half-life and bioavailability. Compositions may also comprise incorporation of soluble AGP-3 R-related protein into liposomes, microemulsions, micelles or vesicles for controlled delivery over an extended period of time. Soluble AGP-3 R-related protein may be formulated into microparticles suitable for pulmonary administration.

Compositions of the invention may be administered by injection (either subcutaneous, intravenous or intramuscular) or by oral, nasal, pulmonary or rectal administration. The route of administration eventually chosen will depend upon a number of factors and may be ascertained by one of ordinary skill in the art.

The invention also provides for pharmaceutical compositions comprising a therapeutically effective amount of the nucleic acids of the invention together with a pharmaceutically acceptable adjuvant. Nucleic acid compositions will be suitable for the delivery of part or all of the coding region of AGP-3R-related protein and/or flanking regions to cells and tissues as part of an anti-sense therapy regimen.

Pharmaceutical Methods of Use

AGP-3R-related proteins and agonists or antagonists thereof may be used to treat conditions characterized by B cell growth, survival, and activation, such as autoimmune and inflammatory disorders. The invention also encompasses modulators (agonists and antagonists) of AGP-3R-related protein and the methods for obtaining them. Such a modulator may either increase or decrease at least one form of AGP-3 related activity, such as B cell growth, survival, or activation in MLN, spleen, and Peyer's patches. Typically, an agonist or antagonist may be a co-factor, such as a protein, peptide, carbohydrate, lipid or small molecular weight molecule, that interacts with AGP-3R and regulates activity. Potential polypeptide antagonists include antibodies that react with AGP-3R, a soluble form of AGP-3R, fusion proteins comprising a soluble form of AGP-3R, and derivatives of soluble AGP-3R. Molecules that regulate AGP-3R-related protein expression typically include nucleic acids that are complementary to nucleic acids encoding AGP-3R-related protein or a fragment thereof and that act as anti-sense regulators of expression.

AGP-3R-related proteins and modulators thereof may be particularly useful in treatment of inflammatory conditions of the joints. Inflammatory conditions of a joint are chronic joint diseases that afflict and disable, to varying degrees, millions of people worldwide. Rheumatoid arthritis is a disease of articular joints in which the cartilage and bone are slowly eroded away by a proliferative, invasive connective tissue called pannus, which is derived from the synovial membrane. The disease may involve peri-articular structures such as bursae, tendon sheaths and tendons as well as extra-articular tissues such as the subcutis, cardiovascular system, lungs, spleen, lymph nodes, skeletal muscles, nervous system (central and peripheral) and eyes (Silberberg (1985), Anderson's Pathology, Kissane (ed.), II:1828). Osteoarthritis is a common joint disease characterized by degenerative changes in articular cartilage and reactive proliferation of bone and cartilage around the joint. Osteoarthritis is a cell-mediated active process that may result from the inappropriate response of chondrocytes to catabolic and anabolic stimuli. Changes in some matrix molecules of articular cartilage reportedly occur in early osteoarthritis (Thonar et al. (1993), Rheumatic disease clinics of North America, Moskowitz (ed.), 19:635-657 and Shinmei et al. (1992), Arthritis Rheum., 35:1304-1308). AGP-3, AGP-3R and modulators thereof are believed to be useful in the treatment of these and related conditions.

AGP-3R-related proteins, and agonists or antagonists of either may also be useful in treatment of a number of additional diseases and disorders, including:

acute pancreatitis;

ALS;

Alzheimer's disease;

asthma;

atherosclerosis;

cachexia/anorexia;

chronic fatigue syndrome;

diabetes (e.g., insulin diabetes);

fever;

glomerulonephritis;

graft versus host disease;

hemorrhagic shock;

hyperalgesia;

inflammatory bowel disease;

inflammatory conditions of a joint, including osteoarthritis, psoriatic arthritis and rheumatoid arthritis;

inflammatory conditions resulting from strain, sprain, cartilage damage, trauma, orthopedic surgery, infection or other disease processes;

ischemic injury, including cerebral ischemia (e.g., brain injury as a result of trauma, epilepsy, hemorrhage or stroke, each of which may lead to neurodegeneration);

learning impairment;

lung diseases (e.g., ARDS);

multiple myeloma;

multiple sclerosis;

myelogenous (e.g., AML and CML) and other leukemias;

myopathies (e.g., muscle protein metabolism, esp. in sepsis);

neurotoxicity (e.g., as induced by HIV);

osteoporosis;

pain;

Parkinson's disease;

pre-term labor;

psoriasis;

reperfusion injury;

septic shock;

side effects from radiation therapy;

sleep disturbance;

temporal mandibular joint disease; and

tumor metastasis.

Agonists and antagonists of AGP-3R-related protein may be administered alone or in combination with a therapeutically effective amount of other drugs, including analgesic agents, disease-modifying anti-rheumatic drugs (DMARDs), non-steroidal anti-inflammatory drugs (NSAIDs), and any immune and/or inflammatory modulators. Thus, agonists and antagonists of AGP-3R-related protein may be administered with:

Modulators of other members of the TNF/TNF receptor family, including TNF antagonists, such as etanercept (Enbrel™), sTNF-RI, D2E7, and Remicade™.

Nerve growth factor (NGF) modulators.

IL-1 inhibitors, including IL-1ra molecules such as anakinra (Kineret™) and more recently discovered IL-1ra-like molecules such as IL-1Hy1 and IL-1Hy2; IL-1 “trap” molecules as described in U.S. Pat. No. 5,844,099, issued Dec. 1, 1998; IL-1 antibodies; solubilized IL-1 receptor, and the like.

IL-6 inhibitors (e.g., antibodies to IL-6).

IL-8 inhibitors (e.g., antibodies to IL-8).

IL-18 inhibitors (e.g., IL-18 binding protein, solubilized IL-18 receptor, or IL-18 antibodies).

Interleukin-1 converting enzyme (ICE) modulators.

insulin-like growth factors (IGF-1, IGF-2) and modulators thereof.

Transforming growth factory-β(TGF-β), TGF-β family members, and TGF-β modulators.

Fibroblast growth factors FGF-1 to FGF-10, and FGF modulators.

Osteoprotegerin (OPG), OPG analogues, osteoprotective agents, and bone anabolic agents.

PAF antagonists.

Keratinocyte growth factor (KGF), KGF-related molecules (e.g., KGF-2), and KGF modulators.

COX-2 inhibitors, such as Celebrex™ and Vioxx™.

Prostaglandin analogs (e.g., E series prostaglandins).

Matrix metalloproteinase (MMP) modulators.

Nitric oxide synthase (NOS) modulators, including modulators of inducible NOS.

Modulators of glucocorticoid receptor.

Modulators of glutamate receptor.

Modulators of lipopolysaccharide (LPS) levels.

Anti-cancer agents, including inhibitors of oncogenes (e.g., fos, jun) and interferons.

Noradrenaline and modulators and mimetics thereof.

Assay Methods of Use

AGP-3 R-related proteins may be used in a variety of assays for detecting agonists, antagonists and characterizing interactions with AGP-3R-related proteins. In general, the assay comprises incubating AGP-3R-related protein under conditions that permit measurement of AGP-3-related activity as defined above. Qualitative or quantitative assays may be developed. Assays may also be used to identify new AGP-3R agonists or antagonists and AGP-3R protein family members.

Binding of natural or synthesized receptor, agonist, or antagonist to AGP-3R-related protein may be carried out in several formats, including cell-based binding assays, membrane binding assays, solution-phase assays and immunoassays. In general, trace levels of a labeled binding molecule are incubated with AGP-3R-related protein samples for a specified period of time followed by measurement of bound molecule by filtration, electrochemiluminescent (ECL, ORIGEN system by IGEN), cell-based or immunoassays. Homogeneous assay technologies for radioactivity (SPA; Amersham) and time-resolved fluoresence (HTRF, Packard) can also be implemented. Binding is detected by labeling a binding molecule (e.g., an anti-AGP-3R antibody) with radioactive isotopes (125I, 35S, 3H), fluorescent dyes (fluorescein), lanthanide (Eu³⁺) chelates or cryptates, orbipyridyl-ruthenium (Ru²⁺) complexes. It is understood that the choice of a labeled probe will depend upon the detection system used. Alternatively, a binding molecule may be modified with an unlabeled epitope tag (e.g., biotin, peptides, His₆, myc) and bound to proteins such as streptavidin, anti-peptide or anti-protein antibodies that have a detectable label as described above.

Binding molecules in such assays may be nucleic acids, proteins, peptides, carbohydrates, lipids or small molecular weight organic compounds. The binding molecule may be substantially purified or present in a crude mixture. The binding molecules may be further characterized by their ability to increase or decrease AGP-3-related activity in order to determine whether they act as an agonist or an antagonist.

In an alternative method, AGP-3 R-related protein may be assayed directly using polyclonal or monoclonal antibodies to AGP-3R-related proteins in an immunoassay. Additional forms of AGP-3R-related proteins containing epitope tags as described above may be used in solution and immunoassays.

AGP-3R-related proteins are also useful for identification of intracellular proteins that interact with their respective cytoplasmic domains by a yeast two-hybrid screening process. As an example, hybrid constructs comprising DNA encoding the N-terminal 50 amino acids of an AGP-3R-related protein fused to a yeast GAL4-DNA binding domain may be used as a two-hybrid bait plasmid. Positive clones emerging from the screening may be characterized further to identify interacting proteins. This information may help elucidate an intracellular signaling mechanism associated with AGP-3-related activity and provide intracellular targets for new drugs that modulate inflammatory and immune-related diseases and conditions.

A variety of assays may be used to measure the interaction of AGP-3R-related proteins and agonists, antagonists, or other ligands in vitro using purified proteins. These assays may be used to screen compounds for their ability to increase or decrease the rate or extent of binding to AGP-3R-related proteins. In one type of assay, AGP-3R-related protein can be immobilized by attachment to the bottom of the wells of a microtiter plate. A radiolabeled binding molecule and a test molecule can then be added either one at a time (in either order) or simultaneously to the wells. After incubation, the wells can be washed and counted using a scintillation counter for radioactivity to determine the extent of binding to AGP-3R-related protein. Typically, molecules will be tested over a range of concentrations, and a series of control wells lacking one or more elements of the test assays can be used for accuracy in evaluation of the results. An alternative to this method involves reversing the “positions” of the proteins; i.e., immobilizing a binding molecule to the mictrotiter plate wells, incubating with the test compound and radiolabeled AGP-3 related protein, and determining the extent of binding. See, for example, chapter 18 of Current Protocols in Molecular Biology (1995) (Ausubel et al., eds.), John Wiley & Sons, New York, N.Y.

As an alternative to radiolabeling, AGP-3 R-related proteins or a binding molecule may be conjugated to biotin and the presence of biotinylated protein can then be detected using streptavidin linked to an enzyme, such as horseradish peroxidase (HRP) or alkaline phosphatase (AP), that can be detected colorimetrically, or by fluorescent tagging of streptavidin. An antibody directed to AGP-3R-related protein or a binding molecule that is conjugated to biotin may also be used and can be detected after incubation with enzyme-linked streptavidin linked to AP or HRP.

AGP-3R-related proteins or binding molecules may also be immobilized by attachment to agarose beads, acrylic beads or other types of such inert substrates. The substrate-protein complex can be placed in a solution containing the complementary protein and the test compound; after incubation, the beads can be precipitated by centrifugation, and the amount of binding between the AGP-3R-related protein and a binding molecule can be assessed using the methods described above. Alternatively, the substrate-protein complex can be immobilized in a column and the test molecule and complementary molecule passed over the column. Formation of a complex between AGP-3R-related protein and the binding molecule can then be assessed using any of the techniques set forth above (i.e., radiolabeling, antibody binding, and the like).

Another useful in vitro assay is a surface plasmon resonance detector system, such as the Biacore assay system (Pharmacia, Piscataway, N.J.). The Biacore system may be carried out using the manufacturer's protocol. This assay essentially involves covalent binding of either AGP-3 R related protein or a binding molecule to a dextran-coated sensor chip that is located in a detector. The test compound and the other complementary protein can then be injected into the chamber containing the sensor chip either simultaneously or sequentially and the amount of complementary protein that binds can be assessed based on the change in molecular mass that is physically associated with the dextran-coated side of the of the sensor chip; the change in molecular mass can be measured by the detector system.

In vitro assays such as those described above may be used advantageously to screen rapidly large numbers of compounds for effects on complex formation with AGP-3 R-related proteins. The assays may be automated to screen compounds generated in phage display, synthetic peptide and chemical synthesis libraries.

Compounds that increase or decrease complex formation among AGP-3R-related proteins and binding molecules may also be screened in cell culture using cells and cell lines bearing such ligands. Cells and cell lines may be obtained from any mammal, but preferably will be from human or other primate, canine, or rodent sources. Such cells may be enriched from other cell types by affinity chromatography using publicly available procedures. Attachment of AGP-3R-related protein to such cells is evaluated in the presence or absence of test compounds and the extent of binding may be determined by, for example, flow cytometry using a biotinylated antibody. Cell culture assays may be used advantageously to further evaluate compounds that score positive in protein binding assays described above.

Description of Working Examples/Preferred Embodiments

The following examples are offered to illustrate the invention, but should not be construed as limiting the scope thereof.

Materials and Methods

Cloning of Human AGP-3

A TNF family profile search of the Genbank dbEST data base was performed. Smith et al.(1994), Cell, 76: 959-62; Luethy et al.(1994), Protein Science, 3: 139-46. One human EST sequence (GenBank accession number T87299) was identified as a possible new member of the TNF ligand. The EST was obtained from human fetal liver spleen cDNA library (The WashU-Merck EST Project). The cDNA clone (115371 3′) corresponding to the EST sequence was obtained from Genome Systems, Inc. (St. Louis, Mo.). The cDNA fragment was released from the pT7T3D vector with EcoRI and NotI digestion. The fragment was approximately 0.7 kb in length and was used for the subsequent full-length cloning.

The ³²P-dCTP-labeled T87299 cDNA fragment was used as a probe to screen a human spleen cDNA phage library (Stratagene, La Jolla, Calif.). Recombinant phages were plated onto E. coli strain XL1-blue at approximately 5×10⁴ transformants per 150 mm LB plate. Nitrocellulose filters were lifted from these plates in duplicates. Filters were prehybridized in 5×SSC, 50% deionized formamide, 5×Denhardt's solution, 0.5% SDS, and 100 μg/ml denatured salmon sperm DNA for 2 hours at 42° C. The filters were then hybridized in the same solution with the addition of 5 ng/ml of labeled probe at 42° C. overnight. The filters were first washed in 2×SSC and 0.1% SDS for 10 minutes at RT twice, and then washed in 0.1×SSC and 0.1% SDS at 65° C. for 30 minutes twice. The filters were then exposed to autoradiography with intensifying screens at −80° C. overnight. Positive hybridizing plaques were determined by aligning the duplicate filters, and then picked up for subsequent secondary or tertiary screening till single isolated positive plaque was obtained. From total of one million recombinant phage clones, 8 positive plaques were obtained.

The pBluescript phagemid was excised from phage using the ExAssist™/SOLR™ System according to the manufacturer's description (Stratagene, La Jolla, Calif.). The excised phagemids were plated onto freshly grown SOLR cells on LB/ampicillin plates and incubated overnight. Single bacteria colony was amplified in LB media containing 100 μg/ml ampicillin. The plasmid DNA was prepared and both strands of cDNA insert were sequenced.

The human AGP-3 cDNA (clone 13-2) is 1.1 kb in length. It encodes a LORF of 285 amino acids. FASTA search of the SwissProt database with the predicted AGP-3 protein sequence indicated that it is mostly related to human TNFα with 25% identity in C-terminal 116 amino acid overlap. Like other TNF ligand family members, human AGP-3 protein is a type II transmembrane protein, containing a short N-terminal intracellular domain (amino acids 1-46), a hydrophobic transmembrane region (amino acids 47-68) following by a long C-terminal extracellular domain (amino acid 69-285). The C-terminal extracellular domain of AGP-3 contained most of the conserved region of the TNF ligand family. Smith et al.(1994), Cell, 76: 959-62.

Cloning of Murine AGP-3

An EST sequence (Genebank accession number AA254047) encoding a potential murine AGP-3 ortholog was identified by BLAST search of Genebank dbEST database with human AGP-3 sequence. The corresponding cDNA clone (722549 5′) from mouse lymph node library was obtained from Genome Systems, Inc. (St. Louis, Mo.). The clone contained a 0.9 kb cDNA insert which could be released by EcoRI and NotI digestion. The 0.9 kb cDNA fragment encodes an open reading frame of 96 amino acids which shares 87% identity with the corresponding C-terminal human AGP-3 polypeptide sequence. A 0.41 kb EcoRI-XmnI fragment, which contained 290 bp coding region and 120 bp 3′ non-coding region, was used as probe to screening a mouse spleen cDNA phage library (Stratagene, La Jolla, Calif.) for full length murine AGP-3 cDNA as described above. From one million recombinant phage clones, 6 positive plaques were obtained. The phagemid was excised from phage as described above. The plasmid DNA was prepared and both strands of cDNA insert were sequenced. The murine AGP-3 cDNA (clone S6) encodes a polypeptide of 309 amino acids. Like its human ortholog, murine AGP-3 is also a type II transmembrane region, containing a short N-terminal intracellular domain (amino acid 1-46), a hydrophobic transmembrane region (amino acid 47-68) following by a long C-terminal extracellular domain (amino acid 69-285). The human and murine AGP-3 share 68% amino acid sequence identity overall. However, the C-terminal 142 amino acid sequences share 87% identity between the two species. Preceding the highly conserved C-terminus region, there is an insertion of 30 extra amino acids in the murine AGP-3. Four out of 7 positive phage plaques were independent clones, yet they all shared the same coding sequences.

Expression of Human and Murine AGP-3 mRNA

Multiple human or murine tissue northern blots (Clontech, Palo Alto, Calif.) were probed with ³²P-dCTP labeled human AGP-3 0.7 kb EcoRI-NotI fragment or murine AGP-3 0.41 kb EcoRI-XmnI fragment, respectively. The Northern blots were prehybridized in 5×SSC, 50% deionized formamide, 5×Denhardt's solution, 0.5% SDS, and 100 μg/ml denatured salmon sperm DNA for 2 hours at 42° C. The blots were then hybridized in the same solution with the addition of 5 ng/ml of labeled probe at 42° C. overnight. The filters were first washed in 2×SSC and 0.1% SDS for 10 minutes at RT twice, and then washed in 0.1×SSC and 0.1% SDS at 65° C. for 30 minutes twice. The blots were then exposed to autoradiography. The human tissue northern blot analysis with human AGP-3 probe under stringent conditions revealed predominant AGP-3 transcripts with a related molecular mass of 2.4 kb in peripheral blood leukocytes (FIG. 4A). Weaker expression was also detected in human spleen, lung and small intestine (FIG. 4A). Among murine tissues analyzed, murine AGP-3 mRNA, with a relative molecular mass of 2 kb, was mainly detected in spleen, lung, liver and kidney (FIG. 4B).

Overexpression of Murine AGP-3 in Transgenic Mice

Murine AGP-3 cDNA clone S6 in pBluescript SK(−) in pBluescript was used as template to PCR the entire coding region. T3 primer

5′ AAT TAA CCC TCA CTA AAG GG 3″ SEQ ID NO: 28

was used as 5′ PCR primer. The 3′ end PCR primer, which contained a XhoI site, was

5′ TCT CCC TCG AGA TCA CGC ACT CCA GCA AGT GAG 3′ SEQ ID NO: 29

PCR reactions were carried in a volume of 50 μl with 1 unit of vent DNA polymerase (New England Biolabs) in 20 mM Tris-HCl pH 8.8, 10 mM (NH₄)₂SO₄, 0.1% Triton-X100, 10 μM of each dNTP, 1 μM of each primer and 10 ng of murine AGP-3 cDNA template. Reactions were performed in 94° C. for 45 s, 55° C. for 55 S, and 72° C. for 2 minutes, for a total of 35 cycles. The PCR fragment created a XhoI site at 3′ end after the AGP-3 coding region. The 1 kb PCR fragment was purified by electrophoresis, and digested with XbaI (present in the pBluescript MCS, 80 bp upstream of AGP-3 starting Methione) and XhoI restriction enzymes. The XbaI-XhoI PCR fragment was cloned into expression vector under the control of the human β-actin promoter. Graham et al.(1997), Nature Genetics 17: 272-4; Ray et al.(1991), Genes Dev. 5: 2265-73. The PCR fragment was sequenced to ensure no mutation. The murine AGP-3 expression plasmid was purified through two rounds of CsCl density gradient centrifugation. The purified plasmid was digested with ClaI, and a 6 kb fragment containing murine AGP-3 transgene was purified by gel electrophoresis. The purified fragment was resuspended in 5 mM Tris, pH 7.4, 0.2 mM EDTA at 2 μg/ml concentration. Single-cell embryos from BDF1×BDF1-bred mice were injected as described (WO 97/23614). Embryos were cultured overnight in a CO₂ incubator and 15-20 2-cell embryos were transferred to the oviducts of pseudopregnant CD1 female mice.

Following term pregnancy, 62 offspring were obtained from implantation of microinjected embryos. The offspring were screened by PCR amplification of the integrated transgene in genomic DNA samples. Ear pieces were digested in 20 μl ear buffer (20 mM Tris, pH8.0, 10 mM EDTA, 0.5% SDS, 500 μg/ml proteinase K) at 55° C. overnight. The sample was diluted with 200 μl of TE, and 2 μl of the ear sample was used for the PCR reaction. The 5′ PCR primer

| | |
|---|---|
| 5′ AAC AGG CTA TTT CTT CAT CTA CAG 3′ | SEQ ID |
| | NO: 30 |

resided in the murine AGP-3 coding region. The 3′ PCR primer

| | |
|---|---|
| 5′ CTC ATC AAT GTA TCT TAT CAT GTC T 3′ | SEQ ID |
| | NO: 31 |

resided in the vector 3′ to the murine AGP-3 transgene. The PCR reactions were carried in a volume of 50 μl with 0.5 unit of Tag DNA polymerase (Boehringer Mannheim, Indianapolis, Ind.) in 10 mM Tris-HCl pH 8.3, 50 mM KCl, 2.5 mM MgCl₂, 10 μM of each dNTP, 1 μM of each primer and 2 μl of ear sample. The mixtures were first heated at 94° C. for 2 min, and the PCR reactions were performed in 94° C. for 30 s, 55° C. for 30 s, and 72° C. for 45 s, for a total of 35 cycles. Of the 62 offspring, 10 were identified as PCR positive transgenic founders.

At 8 weeks of age, all ten transgenic founders (animal 3, 6, 9, 10, 13, 38, 40, 58, 59, and 62) and five controls (animal 7, 8, 11, 12 and 14) were sacrificed for necropsy and pathological analysis. Portions of spleen were removed, and total cellular RNA was isolated from the spleens of all the transgenic founders and negative controls using the Total RNA Extraction Kit (Qiagen Inc., Chartsworth, Calif.). The expression of the transgene was determined by RT-PCR. The cDNA was synthesized using the SuperScript™ Preamplification System according to the manufacturer's instructions (Gibco BRL, Gaithersburg, Md.). The primer

| | |
|---|---|
| 5′ CTC ATC AAT GTA TCT TAT CAT GTC T 3′ | SEQ ID |
| | NO: 32 |

which was located in the expression vector sequence 3′ to the AGP-3 transgene, was used to prime cDNA synthesis from the transgene transcripts. Ten μg total spleen RNA from transgenic founders and controls were incubated with 1 μM of primer at 70° C. for 10 min, and placed on ice. The reaction was then supplemented with 10 mM Tris-HCl pH 8.3, 50 mM KCL, 2.5 mM MgCl₂, 10 μM of each dNTP, 0.1 mM DTT and 200 U SuperScript II RT. After incubation at 42° C. for 50 min, the reaction was stopped by heating at 72° C. for 15 min. Total RNA were digested by addition of 2 U RNase II and incubation at 37° C. for 20 min. Subsequent PCR reactions were carried out by using murine AGP-3 specific primers. The 5′ PCR primer was

| | |
|---|---|
| 5′ AGC CGC GGC CAC AGG AAC AG 3′ | SEQ ID NO: 33 |

The 3′ PCR primer was

5′ TGG ATG ACA TGA CCC ATA G 3′ SEQ ID NO: 34

The PCR reaction was performed in a volume of 50 μl with 0.5 unit Tag DNA polymerase in 10 mM Tris-HCl pH 8.3, 50 mM KCL, 2.5 mM MgCl₂, 10 μM of each dNTP, 1 μM of each primer and 1 μl of cDNA product. The reaction was performed at 94° C. for 30 s, 55° C. for 30 S, and 72° C. for 1 min, for a total of 35 cycles. The PCR product was analyzed by electrophoresis. Transgene expression was detected in the spleen of all ten AGP-3 transgenic mice founders.

Biological Activity of AGP-3

Prior to euthanasia, all animals were weighed, anesthetized by isofluorane and blood was drawn by cardiac puncture. The samples were subjected to hematology and serum chemistry analysis. The serum globulin level in all the AGP-3 transgenic mice (animal 3, 6, 9, 10, 13, 38, 40, 58, 59 and 62) increased more than 100% as compared to the control littermates (animal 7, 8, 11, 12 and 14, Table 1). Total protein level also increased correspondingly in the transgenic group, while albumin level remained the same. No significant differences in other serum chemistry or hematology parameters were observed at this age.

Radiography was performed after terminal exsanguination. There was no difference in the radiodensity or radiologic morphology of the skeleton. Upon gross dissection, major visceral organs were subject to weight analysis. The spleen weight relative to the body weight increased by approximately 45% in the AGP-3 transgenic group as compared to the control mice. The sizes of lymph nodes and Peyer's patches were also increased substantially in all the AGP-3 transgenic mice.

Following gross dissection, tissues were removed and fixed in 10% buffered Zn-Formalin for histological examination. The tissues collected were liver, spleen, pancreas, stomach, the entire gastrointestinal tract, kidney, reproductive organs, skin and mammary glands, bone, brain, heart, lung, thymus, trachea, esophagus, thyroid, adrenals, urinary bladder, lymph nodes and skeletal muscle. After fixation, the tissues were processed into paraffin blocks, and 3 μm sections were obtained. All sections were stained with hematoxylin and exosin, and subject to histologic analysis. The size and the number of the follicles in the spleen, lymph nodes and Peyer's patches were increased significantly in the AGP-3 transgenic mice (FIGS. 5, 6 and 7). The spleen, lymph node and Peyer's patches of both the transgenic and the control mice were subject to immunohistology analysis with B cell and T cell specific antibodies. The formalin fixed paraffin embedded sections were deparaffinized and hydrated to deionized water. The sections were quenched with 3% hydrogen peroxide, blocked with Protein Block (Lipshaw, Pittsburgh, Pa.), and incubated in rat monoclonal anti-mouse B220 and CD3 (Harlan, Indianapolis, Ind.), respectively. The binding was detected by biotinylated rabbit anti-rat immunoglobulins and peroxidase conjugated streptavidin (BioGenex, San Ramon, Calif.) with DAB as chromagen (BioTek, Santa Barbara, Calif.). Sections were counterstained with hematoxylin. The B cell numbers, as indicated by positive B220 staining, increased significantly in the spleen, lymph nodes and Peyer's batches (FIGS. 5, 6, and 7). The T cell numbers, as indicated by the anti-CD3 staining, were slightly decreased. There were no differences in the morphology of the thymus between the transgenic and the control group. By immunohistology, the T cell population was similar in numbers. At 8 weeks of age, there are no distinctive morphologic changes in the liver, kidneys, or urinary, central nervous, hematopoietic, skeletal, respiratory, gastrointestinal, endocrine, or reproductive systems.

After necropsy, MLN and sections of spleen and thymus from 10 AGP-3 transgenic mice (animals 3, 6, 9, 10, 13, 38, 40, 58, 59 and 62) and 5 control littermates (animals 7, 8, 11, 12, and 14) were removed. Single cell suspensions were prepared by gently grinding the tissues with the flat end of a syringe against the bottom of a 100 μm nylon cell strainer (Becton Dickinson, Franklin Lakes, N.J.). Cells were washed twice in a 15 ml volume then counted. Approximately 1 million cells from each tissue was stained with 0.5 μg antibody in a 100 μl volume of PBS (without Calcium and Magnesium)+0.1% Bovine Albumin+0.01% Sodium Azide. All spleen and MLN samples were incubated with 0.5 μg CD16/32(FcγIII/II) Fc block in a 20 μl volume for 10 minutes prior to the addition of FITC or PE-conjugated monoclonal antibodies against CD90.2 (Thy-1.2), CD45R (B220), CD11b(Mac-1), Gr-1, CD4, or CD8 (PharMingen, San Diego, Calif.) at 2-8° C. for 30 min. The cells were washed then analyzed by flow cytometry using a FACScan (Becton Dickinson, San Jose, Calif.). Thymus samples were stained with FITC conjugated anti-Thy-1.2, FITC conjugated anti-CD4, and PE conjugated anti-CD8 (PharMingen, San Diego, Calif.).

In the MLN of the AGP-3 transgenic mice, the percentage of B220 positive B cells increased by 100% (FIG. 6). The percentage of the Thy-1.2 positive T cells decreased approximately 36%, with similar reductions in both CD4(+) and CD8(+) populations. The helper CD4(+)/suppressor CD8(+) ratio remained unchanged. Similar increases in B cell and reductions in T cell populations were also observed in the spleens of the AGP-3 transgenic mice (FIG. 8), though to a lesser extent. No obvious changes in staining with anti-CD11b or anti-Gr-1 antibodies were observed in the lymph node and spleen between the transgenic and the control group. In the thymus, there were no differences in the percentages of Thy-1.2(+), CD4(+), CD8(+) or CD4(+)CD8(+) populations between the AGP-3 transgenic and the control mice.

Serum Immunoglobulin and Autoantibody Analysis

Transgenic mice and control littermates were bled successively at 6, 7, 8, 9, 11, and 12 weeks of age. Serum immunoglobulin levels were quantitated using by ELISA with Mouse Hybridoma Subtype Kit as suggested by manufacture (Boehringer Mannheim, Indianapolis, Ind.). Presence of autoantibodies directed against nuclear antigens and dsDNA were examined in the serum by enzyme linked immunosorbant assay (ELISA). The levels of anti-nuclear antibodies were detected using ANA screen kit (Sigma) and anti-mouse IgG peroxidase secondary antibody. Mouse serum samples were diluted 1:200 in ANA screen ELISA. For the detection of anti-dsDNA autoantibodies in serum, high binding ELISA plates were coated with plasmid DNA (Immunovision) as an antigen in the presence of methylated BSA. After blocking the non-specific sites and washing, diluted mouse serum samples were added to wells in duplicated and the binding was quantitated using horse radish peroxidase-labeled anti-mouse IgG or anti-mouse IgM reagents (Southern Biotech). A pooled positive serum from BWF1 mice and pooled negative serum from B6 mice was used as controls. Experiment for the detection of anti-histone antibodies was essentially done similar to anti-DNA ELISA except that carbonate-bicarbonate buffer (pH9.6) buffer was used as coating buffer. Serum antibody data were compared by Mann Whitney test using Sigmastat software (SPSS Science, Chicago, Ill.).

B Cell Survival and Proliferation Assay

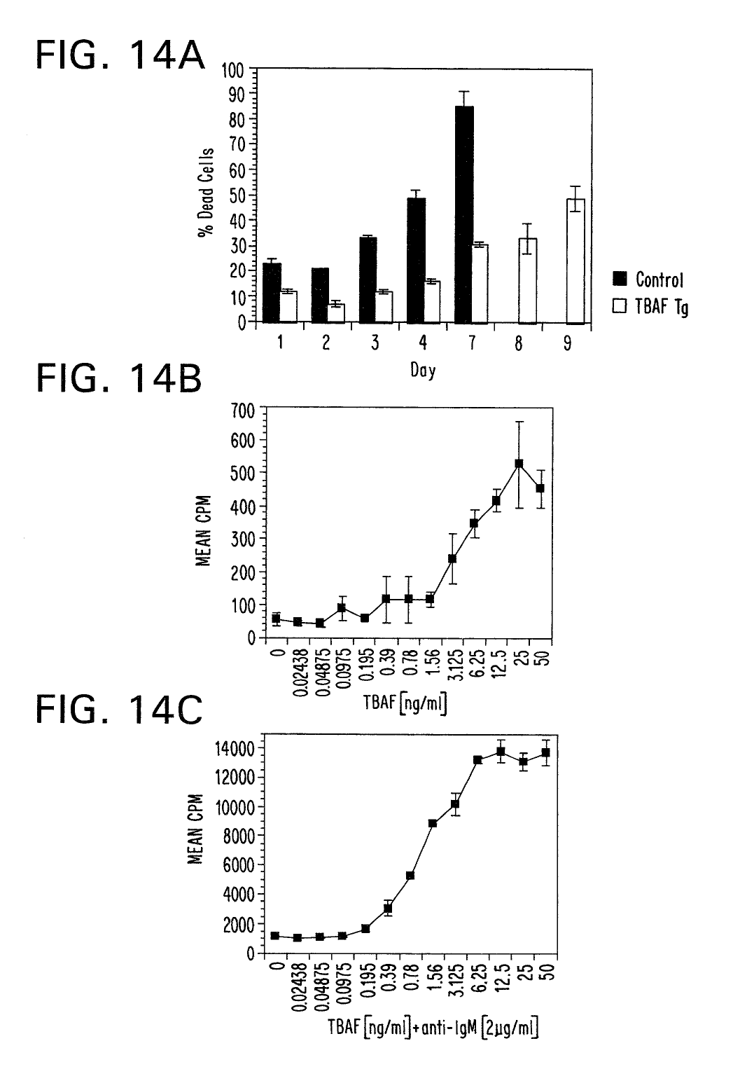

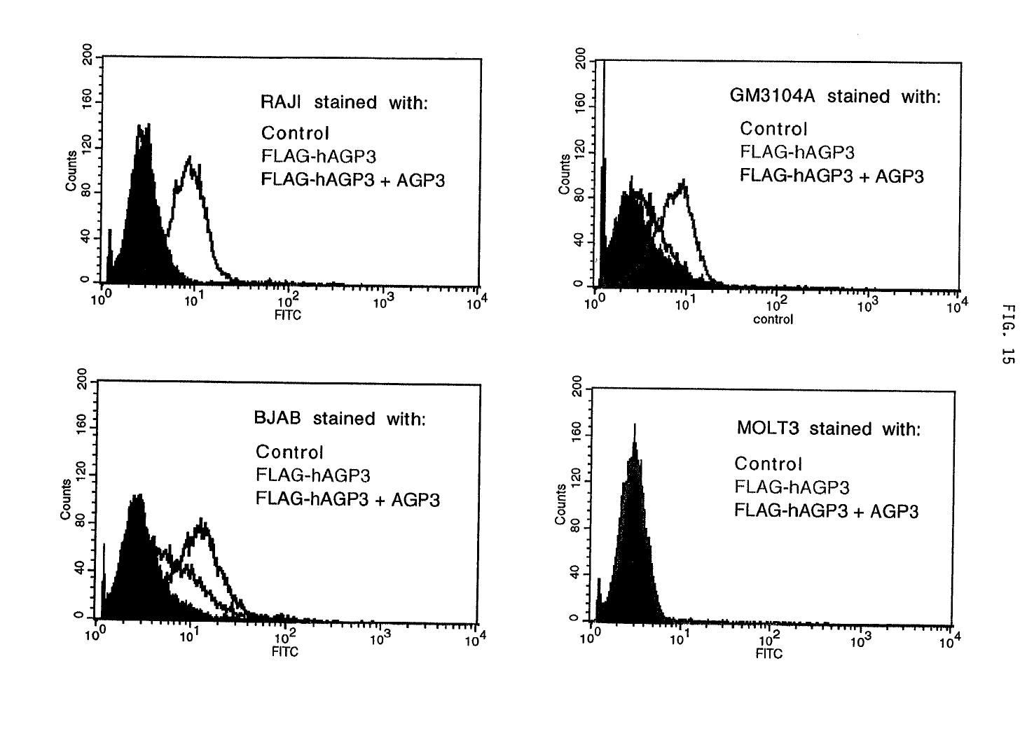

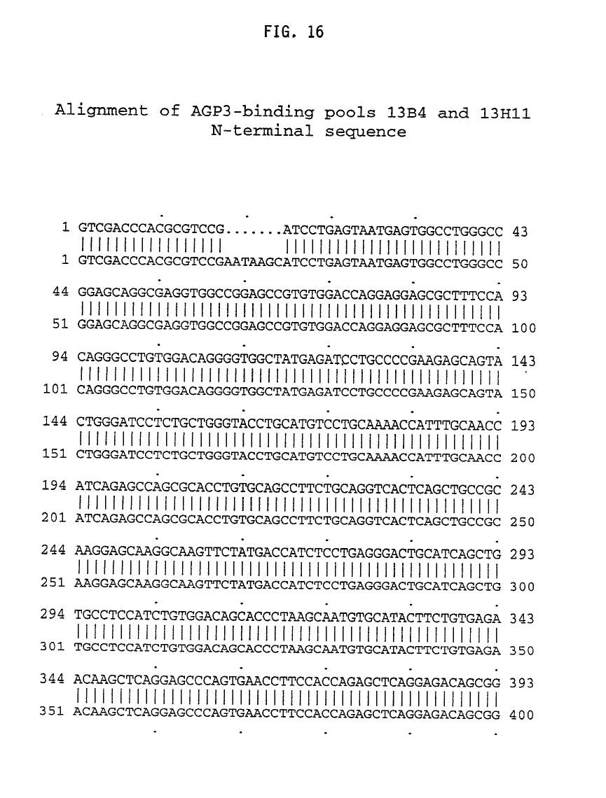

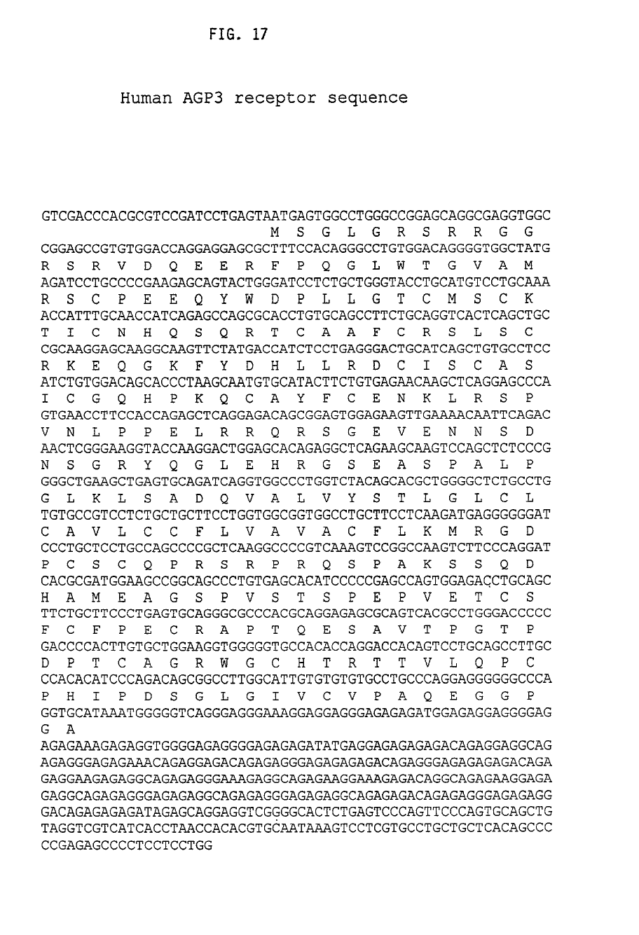

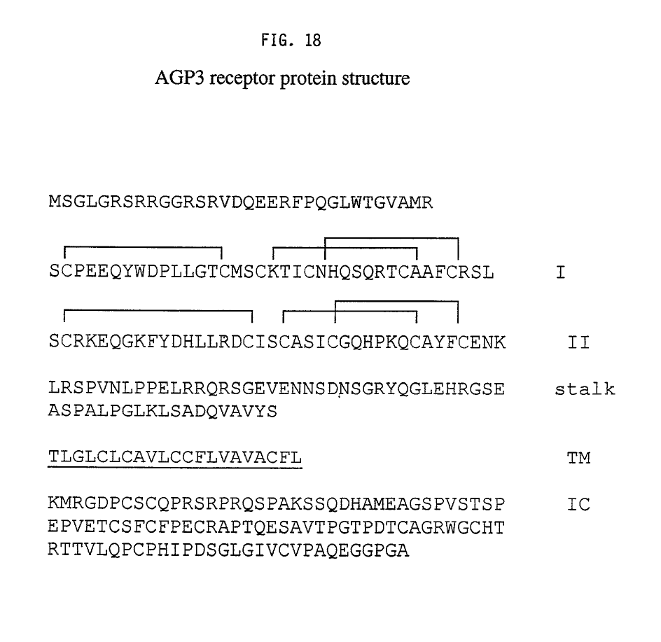

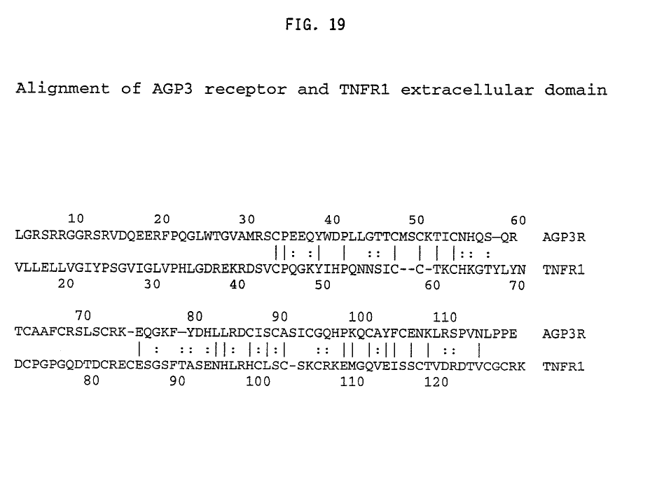

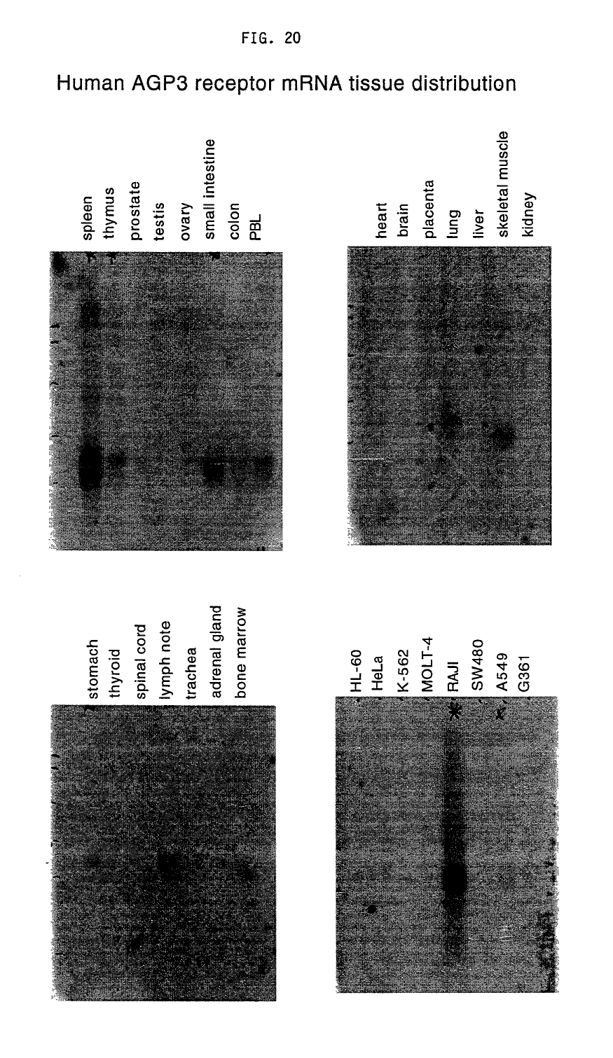

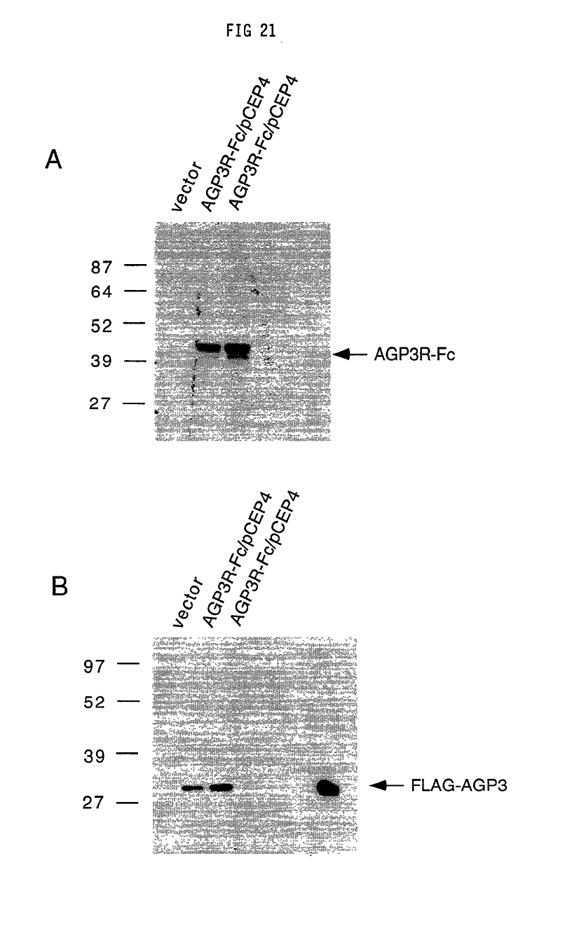

Cells were isolated from spleens of 2-4 months old mice by negative selection. Briefly, B lymphocytes were purified by density gradient centrifugation and then passed over a B cells column (Accurate/Cedarlane, Westbury, N.Y.). Cells isolated by this method were analyzed by flow cytometry and >90% were found positive for B220 staining. Isolated B cells were cultured in MEM+10% FCS at 37° C., 5% CO₂. Cells were collected from triplicate wells daily on day 1 through day 9 and incubated with 5·g/ml Propidium Iodide. Cells were analyzed by Flow cytometry and the percentage of dead cells was calculated. For B cell proliferation assay, purified (10⁵) B cells from B6 mice as described above were cultured in MEM+10% heat inactivated FCS in triplicate in 96 well flat bottomed plate with/without 2 μg/ml of Goat F(ab′)₂ anti-mouse IgM (Jackson ImmunoResearch Laboratory, West Grove, Pa.) and/or indicated amount of recombinant AGP-3 for a period of 4 days at 37° C., 5% CO₂. Proliferation was measured by an uptake of radioactive ³(H) thymidine in last 18 hours of pulse. Data is shown in FIG. 14 as mean±standard deviation of triplicate wells.

B Cell Hyperplasia and Hypergammaglobulinemia in AGP-3Transgenic Mice

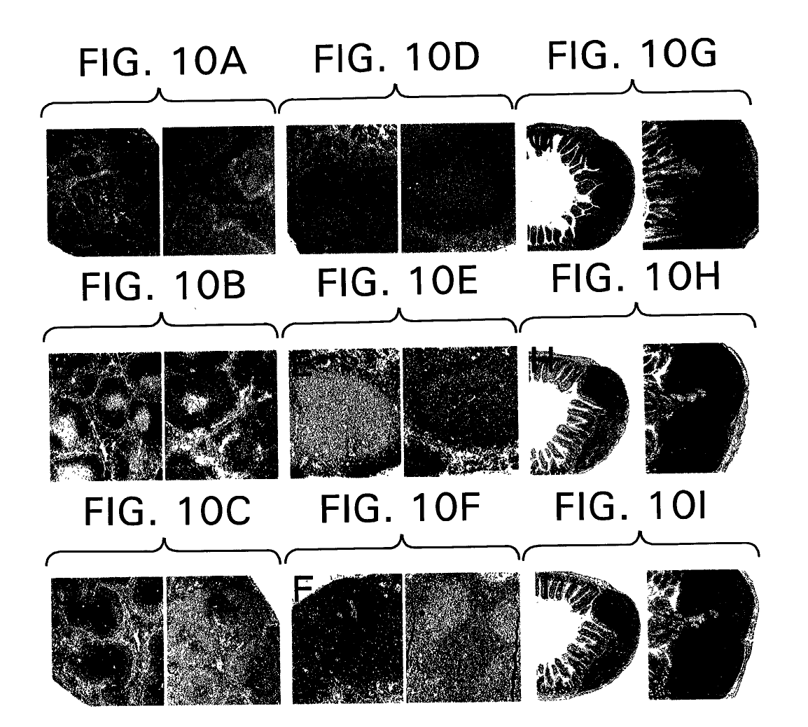

To gain insights into the biological function for AGP-3, transgenic mice were generated that expressed full-length murine AGP-3 protein driven by the ubiquitous 3-actin promoter. Founder mice harboring the AGP-3 transgene were identified by PCR analysis of genomic DNA samples. Transgene expression was confirmed by RT-PCR from spleen total RNA. At 8 weeks of age, ten AGP-3 transgenic mice and five control littermates were subject to necropsy and pathological analysis. The transgenic mice were of normal size and weight. However, the spleen weight relative to the body weight increased by approximately 45% in the AGP-3 transgenic group as compared to the control mice. The sizes of lymph nodes and Payer's Patches were also increased substantially in all the AGP-3 transgenic mice. Histology analysis demonstrated that the size and the number of the follicles in the spleen, lymph nodes and Payer's patches were increased significantly in the AGP-3 transgenic group (FIG. 10). Immunohistology staining with B and T cell specific markers indicated the B cell numbers increased significantly in the spleen, lymph nodes and Payer's patches of the transgenic group (FIG. 10). The T cell numbers, as indicated by the anti-CD3 staining, were decreased correspondingly (FIG. 10). There were no differences in the morphology and immunostaining of thymus between the transgenic and the control groups. No changes were observed in other organs or organ systems of the 8 weeks old transgenic mice, including kidney, liver, and hematopoietic tissues.

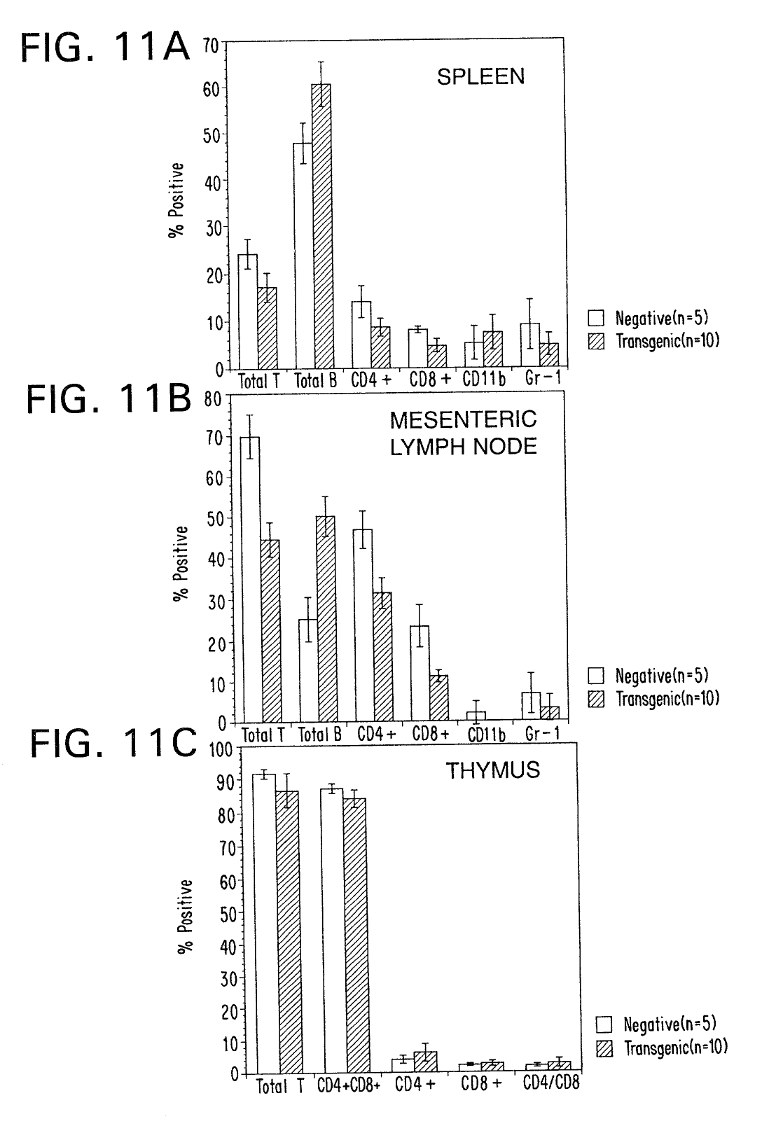

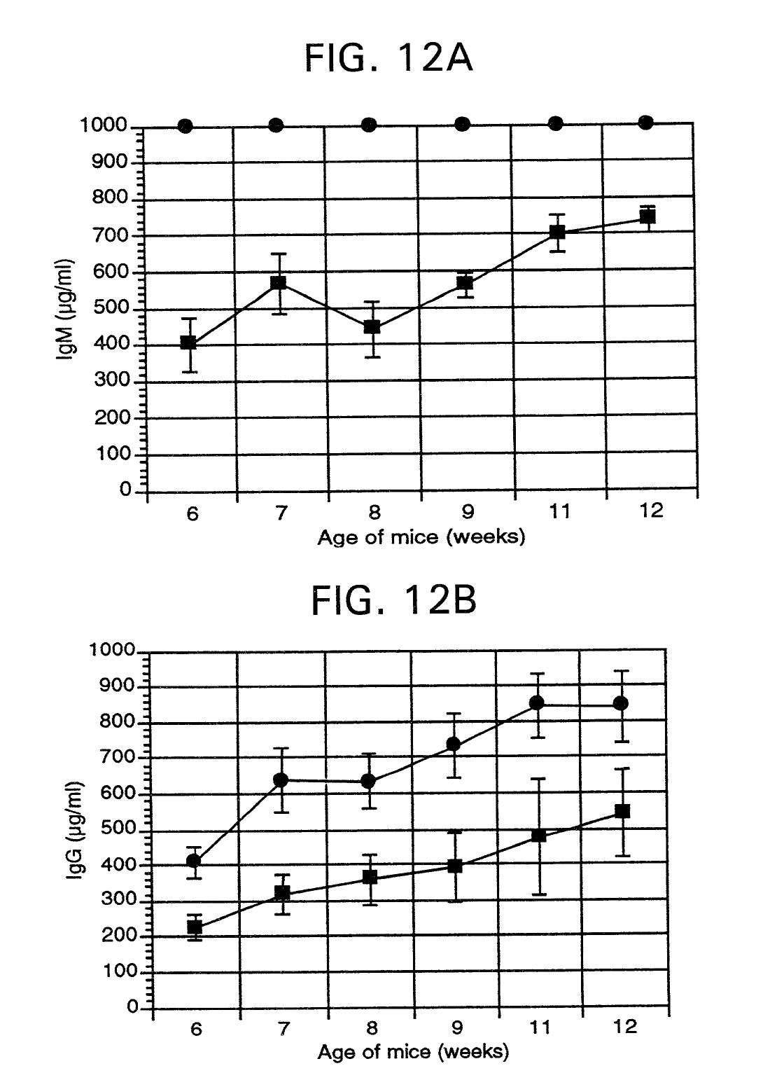

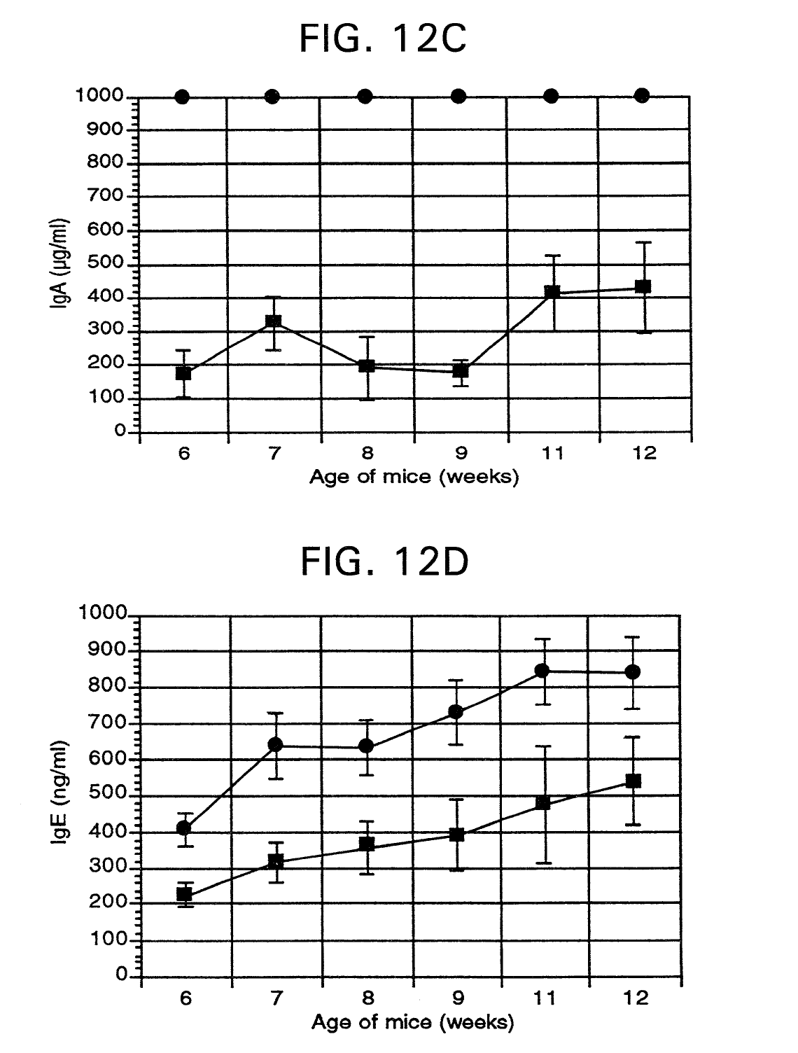

The B cell hyperplasia phenotype in the AGP-3 transgenic mice was also confirmed by flow cytometry analysis. In the mesenteric lymph nodes of the AGP-3 transgenic mice, the percentage of B220 positive B cells increased by 100% (FIG. 11). The percentage of the Thy-1.2 positive T cells decreased by approximately 36%, with similar reductions in both CD4(+) and CD8(+) T cells. Similar increase in B cell and reduction in T cell populations were also observed in the spleens of the AGP-3 transgenic mice, though to a lesser extent (FIG. 11). Of note, the total T cell numbers in the lymph node and spleen of AGP-3 transgenic mice were similar to the control littermates. In the thymus, there were no differences in the percentages of single positive CD4(+) or CD8(+) T cells, or CD4(+)CD8(+) populations between the AGP-3 transgenic and the control mice (FIG. 11). No obvious changes in staining with anti-CD11b or anti-Gr-1 antibodies were observed in the lymph nodes and spleen between the transgenic and the control group (FIG. 11). The histological and FACS analysis, together, suggested severe B cell hyperplasia phenotype in the AGP-3 transgenic mice.

We also examined B cell populations of different developmental stages by FACS analysis. No differences were observed in the percentage of the pro B (B220+IgM−), immature B (B220+IgM+), or mature B (IgM+IgD+) within spleenic B cell population of the AGP-3 transgenic mice as compared to the control littermates. In addition, the number of the spleenic CD5+B cells in the AGP-3 transgenic mice from 1 to 9 month of age was unaltered. We also didn't detect any alteration of the CD40 expression level on B cells in the transgenic mice, suggesting that the B cell hyperplasia in the AGP-3 transgenic mice was not caused by CD40 upregulation.

In addition to the B cell hyperplasia phenotype, the AGP-3 transgenic mice also had severe hypergammaglobulinemia. The serum globulin level in AGP-3 transgenic mice increased more that 100% as compared to the control group. Total protein level also increased correspondingly in the transgenic, while albumin level remained the same. The increased B cell numbers and high serum globulin level suggested elevated serum immunoglobulin titer. Thus we examined serum levels of IgM, IgG, IgA and IgE of AGP-3 transgenic mice from 6 to 12 weeks of age. Comparing to the same age control littermates, serum IgM, IgG, IgA and IgE were significantly increased in all age groups of AGP-3 transgenic mice. The increase found in serum IgG was not specific to any particular subclass (IgG1, IgG2a, IgG2b, and IgG3). No significant differences in other serum chemistry or hematology parameters were observed at this age. The increased serum immunoglobulin levels is likely to result directly from increased B cell number, but may also be aggravated by increased B cell antibody production.

Autoantibodies Associated with Lupus in AGP-3 Transgenic Mice

Increased humoral immunity in AGP-3 transgenic mice warranted us to look for possible phenotypes resembling B cell associated autoimmune diseases such as systemic lupus erythematosus (SLE). The common denominator in lupus patients and lupus prone mice is IgG autoantibody production, and the hallmark of this disease is the presence of elevated anti-nuclear antibodies in the serum. The emergence of anti-DNA antibodies represents one final outcome in the different murine lupus models and patients with SLE. When sera from transgenic and non-transgenic mice at various age were examined for the presence of autoantibodies recognizing nuclear antigens or dsDNA, two different lines of AGP-3 transgenic mice began to show presence of autoantibodies at around 8 weeks of age (Table 1). The amount of anti-nuclear and anti-dsDNA antibody increased with their age in the transgenic animals (Table 1). More interestingly, at 5 and 8 months of age, AGP-3 transgenic mice showed 5-10 higher amount of anti-dsDNA antibodies compared to age matched lupus prone (NZB×NZW)F1 mice. The presence of autoantibodies in the serum of AGP-3 transgenic mice did not discriminate between gender of mice. Both IgG and IgM antibodies to dsDNA were detected in transgenic animals. Presence of such autoantibodies was undetectable in non-transgenic littermates, as expected.

Immune Complex Deposits in the Kidney of AGP-3 Transgenic Mice

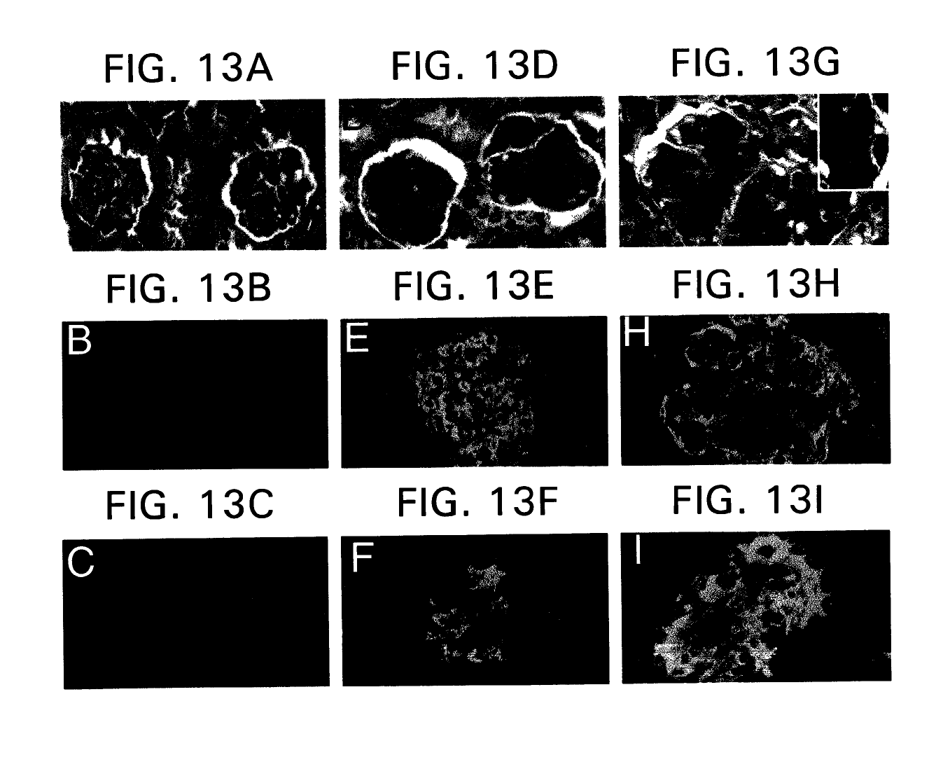

Presence of anti-DNA antibodies followed by immune complex induced renal damage is classical picture seen in lupus associated nephritis. At 5 month of age, the AGP-3 transgenic mice developed glomerular proteinaceous deposits in the kidney (FIG. 13). The deposits were seen in more than 60% of the glomeruli in the transgenic mice, but absent in the control littermates. Immunohistology showed the deposits contained moderate amounts of IgG and larger amounts of IgM (FIG. 13). Trichrome staining showed no deposit of connective tissues in the glomeruli at 5 month of age. There is also no evidence of any cellular proliferation or presence of inflammatory cells at this age (FIG. 13). Interestingly, the kidney lesions progressed as the transgenic mice grew older. At 8 month of age, there was obvious enlargement of glomeruli in the AGP-3 transgenic mice as compared to the age matched control littermates (FIG. 13G). In addition, we also detected extensive connective tissue deposits in the enlarged glomeruli (FIG. 13G). Comparing to the 5 month old mice, the 8 month old transgenic mice had increased IgG level in the glomeruli immune complex deposits (FIG. 13I). Majority of the glomeruli in the AGP-3 transgenic mice were affected. We also performed serum and urine chemistry analysis of 5 month old and 8 month old AGP-3 transgenic along with the control littermates. No significant differences were noticed in the 5 month old AGP-3 transgenic mice. However, in the 8 month old mice, we observed increases in serum blood urea nitrogen (BUN) and calcium levels and decrease in serum phosphate level. In addition, the 8 month old AGP-3 mice also had increased protein level in the urine. These changes, together, suggest the onset of renal failure in the 8 month old AGP-3 transgenic mice. In conclusion, the high serum autoantibodies followed by the kidney lesions in the AGP-3 transgenic mice clearly resemble to the pathological progression in the SLE patients and lupus prone mice.

AGP-3 Stimulates B Cell Survival and Proliferation: a Possible Mechanism for Autoimmunity

The B cell hyperplasia phenotype in the AGP-3 transgenic mice might arise from increased B cell survival and/or increased B cell proliferation. We first compared the viability of B cells from AGP-3 transgenic mice with that of the control littermates. B cells were isolated from both transgenic or control mice and incubated in minimal essential medium supplemented with 10% heat inactivated fetal bovine serum. Viability of the B cells was measured by FACS analysis for Propidium Iodide uptake (FIG. 14A). By day 3, 30% of B cells isolated from the control mice were dead, whereas only 10% of B cells from AGP-3 transgenic mice were dead. By day 5, 70% of B cells from AGP-3 mice were still viable, whereas only 15% of B cell from control littermates were viable. By day 9, almost 50% of the AGP-3 transgenic B cells still remained viable. Therefore, transgenic expression of AGP-3 prolonged B cell viability. It remains to be determined if this B cell survival stimuli result directly from AGP-3 action on B cells or through its modulation of the immune system.

Recently Schneider et al (Schneider et al., 1999, and Moore et al., 1999) reported co-stimulation of B cell proliferation by BAFF/BLYS with anti-IgM. We found that AGP-3 alone can also stimulates B cell proliferation in a dose dependent manner with an ED₅₀ of approximately 3ng/ml (FIG. 14B, upper). A ten fold increase of B cell proliferation was detected by AGP-3 treatment at 10 ng/ml concentration as compared to the untreated cells. In our experiment, anti-IgM alone at 2 μg/ml concentration increased B cell proliferation by 24 fold. Treatment with anti-IgM (2 μg/ml) in combination with various doses of AGP-3 led to dose dependent increase of B cell proliferation, with a maximal 13 fold increase as compared anti-IgM treatment alone and a total of 320 fold increase as compared to the untreated cells. Thus, AGP-3 is a potent B cell stimulatory factor. The increased B cell survival and proliferation may together contribute to the B cell hyperplasia and autoimmune lupus like changes in the AGP-3 transgenic mice.

TABLE 1
Lupus associated autoantibodies in the serum of AGP-3
transgenic mice.

| Auto- | Age | | Non-tg littermates | |
|---|---|---|---|---|
| antibodies | (months) | AGP-3 ta (n) | (n) | p value |
| Antinuclear | 2-3 | 7ˆ (9) | 1*(8) | |
| antibodies | 5-6 | 9 (9) | 1*(8) | |
| (IgG)^{a} | 8-9 | 8 (8) | 1*(6) | |
| Anti-dsDNA | <2 | 697 ± 284 (7) | 277 ± 67 (7) | NS |
| (IgG)^{b} | 3-4 | 842 ± 351 (7) | 235 ± 49 (7) | <.005 |
| | 6-7 | 2515 ± 428 (5) | 970 ± 344 (7) | <.019 |
| | 8-10 | 12293 ± 6767 | 1070 ± 602 (12) | <.017 |
| | | (11) | | |
| Anti-dsDNA | <2 | 275 ± 33 (7) | 46 ± 5 (7) | <.001 |
| (IgM)^{b} | 3-4 | 1684 ± 920 (7) | 63 ± 13 (7) | <.003 |
| | 6-7 | 6998 ± 5515 | 98 ± 14 (7) | <.001 |
| | | (5) | | |
| | 8-10 | 13712 ± 9147 | 79 ± 14 (12) | <.001 |
| | | (11) | | |
| Anti-Histone | <2 | 741 ± 264 (7) | 52 ± 8 (7) | <.001 |
| (Ig)^{b} | 3-4 | 837 ± 436 (7) | 53 ± 14 (7) | <.003 |
| | 6-7 | 4220 ± 933 (5) | 60 ± 10 (7) | <.001 |
| | 8-10 | 16555 ± 4618 | 295 ± 173 (12) | <.001 |
| | | (11) | | |

ˆ includes two weak positive.
* Weak positive
^{a}Data is shown as number of ANA positive (mean + 2sd of transgene negative littermates) mice using ANA screen kit.
^{b}Data is represented as mean ± SE for each group. Values are shown as Units/ml.
NS: not significant

Bacterial Expression of AGP-3 Protein

PCR amplification employing the primer pairs and templates described below are used to generate various forms of human AGP-3 proteins. One primer of each pair introduces a TAA stop codon and a unique XhoI or SacII site following the carboxy terminus of the gene. The other primer of each pair introduces a unique NdeI site, a N-terminal methionine, and optimized codons for the amino terminal portion of the gene. PCR and thermocycling is performed using standard recombinant DNA methodology. The PCR products are purified, restriction digested, and inserted into the unique NdeI and XhoI or SadI sites of vector pAMG21 (ATCC accession no. 98113) and transformed into the prototrophic E. coli 393 or 2596. Other commonly used E. coli expression vectors and host cells are also suitable for expression. After transformation, the clones are selected, plasmid DNA is isolated and the sequence of the AGP-3 binding protein insert is confirmed.

pAMG21-Human AGP-3 Protein [128-285]

This construct was engineered to be C-terminal 158 amino acids of human AGP-3 and have the following N-terminal and C-terminal residues:

NH₂-MNSRNKR -------GALKLL-COOH.

(SEQ ID NOS: 35 and 52, respectively)

The template to be used for PCR was human AGP-3 in pCDNA3.1(+) vector. Oligonucleotides #1761-31 and #1761-33 were the primer pair to be used for PCR and cloning this gene construct.

| | |
|---|---|
| 1761-31: | |
| 5′-ATT TGA TTC TAG AAG GAG GAA TAA CAT ATG AAC AGC CGT AAT AAG | (SEQ ID NO:_36) |
| CGT GCC GTT CAG GGT -3′ | |
| 1761-33: | |
| 5′-CCG CGG ATC CTC GAG TTA GAG CAG TTT CAA TGC ACC AAA AAA TGT | (SEQ ID NO:_37) |
| -3′ | |

pAMG21-Human FLAG-AGP-3 Protein [128-285]

This construct was engineered to be C-terminal 158 amino acids of human AGP-3 preceded with FLAG epitope. The construct encoded following following N-terminal and C-terminal residues:

| | |
|---|---|
| NH₂-MDYKDDDDKKLNSRNKR-------GALKLL-COOH | (SEQ ID NOS: 38 and 52) |

The template to be used for PCR was human AGP-3 in pCDNA3.1(+) vector. Oligonucleotides #1761-32 and #1761-33 were the primer pair to be used for PCR and cloning this gene construct.

| | |
|---|---|
| 1761-32: | |
| 5′-GAC GAT GAC AAG AAG CTT AAC AGC CGT AAT AAG CGT GCC GTT CAG | (SEQ ID NO:39) |
| GGT -3′ | |
| 1761-33: | |
| 5′-CCG CGG ATC CTC GAG TTA CAG CAG TTT CAA TGC ACC AAA AAA TGT | (SEQ ID NO:_38) |
| -3′ | |

E. coli were induced during fermentation, the lysate was applied to Q Sepharose FF (Pharmacia, Piscaataway, N.J.) equilibrated in 10 mM Mes pH 6.0 and eluted with 50-400 mM NaCl gradient over 30 column volumes. Fractions containing AGP-3 were pooled and loaded onto a Q Sepharose HP column (Pharmacia, Piscataway, N.J.) equilibrated in 10 mM Tris-HCL pH 8.5. AGP-3 was eluted with an increasing linear NaCl gradient (50 mM-200 mM) over 30 column volumes. Endotoxin was removed by application to Sp HiTRAP column (Pharmacia, Piscataway, N.J.) pH 4.8 and eluted with 100-500 mM NaCl in 10 mM sodium acetate pH 4.8 over 25 column volumes. Final endotoxin level of the purified protein is approximately 0.2 EU/mg. The purified human AGP-3 is truncated at residue Arg133 as indicated by N-terminal sequencing and has a molecular weight of 16.5 KDa by reducing SDS-PAGE. The purified human FLAG-AGP-3 protein is confirmed by N-terminal sequence analysis of the protein. The FLAG-AGP-3 protein is recognized by M2 monoclonal antibody against FLAG epitope (Kodak, New Haven, Conn.).

For europium labeling of the protein, human AGP-3 (lot# 092299) was dialyzed into 50 mM sodium carbonate pH 9. Europium labeling reagent (Wallac Delfia reagent lot# 704394) was dissolved in the same buffer. AGP-3 protein was mixed with a 20-fold molar excess of labeling reagent for 24 hours at room temperature. The mixture was then placed on a Sephadex G-25 column which had been equilibrated in 50 mM Tris-HCl pH 7.8, 150 mM NaCl. The protein was eluted from the column with the same buffer. Protein concentration was determined using the BCA method (Pierce Chemical Co.).

Expression Cloning of AGP-3 Receptor

AGP-3 induces B cell proliferation and survival, suggesting the presence of its receptor on primary B cells or B cell lines. To identify a source of AGP-3 receptor, recombinant FLAG-AGP-3 protein was used as immunoprobe to screen for its receptor located on the surface of various cell lines and primary hematopoietic cells. Cells were harvested from exponentially replicating cultures in growth media, pelleted by centrifugation, washed with phosphate buffered saline (PBS) (Gibco) containing 1% fetal calf serum (FCS), and then resuspended at 1×10⁷ cells/ml in a 96 well microtiter tissue culture plate (Falcon) in PBS with 1% FCS containing 1 μg/ml FLAG-AGP-3. After 1 hour incubation at 4° C., cells were washed with PBS with 1% FCS, and then incubated in PBS with 1% FCS containing 20 μg/ml anti-FLAG M2 monoclonal antibody (Kodak, New Haven, Conn.) for 30 minutes at 4° C. After washing with PBS, cells were incubated again in PBS with 1% FCS containing 20 μg/ml FITC-conjugated goat anti-mouse IgG (Southern Biotech Associates, Birmingham, Ala.) for 30 minutes at 4° C. After washing, cells were then analyzed by fluorescence activated cell sorting (FACS) using a Becton Dickinson FACscan. The specificity of the binding was confirmed by addition of 100 μg/ml AGP-3 protein during the first incubation period.

Using this approach, human Burkitt lymphoma RAJI and BJAB cells, and human lymphoblast GM3104A were found to express a surface molecule which could be detected by FLAG-AGP-3 (FIG. 15). Secondary antibody alone did not bind to the surface of these cell lines. This binding could be competed in a dose dependent manner by the addition of non-FLAG tagged AGP-3 protein. The binding by FLAG-AGP-3 protein was not detected on human acute T lymphoblastic leukemic Molt3 cells.

A cDNA library was prepared from 32D mRNA, and ligated into a mammalian expression vector. Exponentially growing RAJI were harvested, and total cell RNA was purified by acid guanidinium thiocyanate-phenol-chloroform extraction (Chomczynski and Sacchi. Anal. Biochem. 162, 156-159, (1987)). The poly (A+) mRNA fraction was obtained from the total RNA preparation by adsorption to, and elution from, Dynabeads Oligo (dT)25 (Dynal Corp) using the manufacturer's recommended procedures. A directional, oligo-dT primed cDNA library was prepared using the Superscript Plasmid System (Gibco BRL, Gaithersburg, Md.) using the manufacturer's recommended procedures. The resulting cDNA was digested to completion with Sal I and Not I restriction endonuclease, then fractionated by size exclusion gel chromatography. The highest molecular weight fractions were selected, and then ligated into the polylinker region of the expression vector. This vector contains the CMV promoter upstream of multiple cloning site, and directs high level expression in eukaryotic cells. The library was then electroporated into competent E. coli (ElectroMAX DH10B, Gibco, N.Y.), and titered on LB agar containing 100 μg/ml ampicillin. The library was then arrayed into segregated pools containing approximately 100 clones/pool, and 1.0 ml cultures of each pool were grown for 16-20 hr at 37° C. Plasmid DNA from each culture was prepared using the Qiagen Qiawell 96 Ultra Plasmid Kit (catalog #16191) following manufacturer's recommended procedures.

Arrayed pools of RAJI cDNA expression library were individually transfected into 293 cells (ATCC), then assayed for the acquisition of a cell surface AGP-3 binding protein using MultiPitte™ (Sagian Inc.). To do this, 293 cells were plated at a density of 1.5×10⁴ per ml in 96-well tissue culture plates (Falcon), then cultured overnight in DMEM (Gibco) containing 10% FCS. Approximately 300 ng of plasmid DNA from each pool was diluted into 75 μl of OPTI-MEMI Reduced Serum Medium (Life Technologies, Gaithersburg, Md.). Simultaneously, 1 μl of DMRIE-C (Life Technologies, Gaithersburg, Md.) was added to diluted into 75 μl of OPTI-MEMI Reduced Serum Medium. The DNA and DMRIE-C solutions were mixed, and allowed to incubate at room temperature for 30 min. The 293 cell cultures were exposed to the DNA-DMRIE-C complexes for 2-5 hr at 37° C. After this period, the cells supplemented with equal volume of DMEM containing 20% FCS. The cells were then cultured for 48 hr at 37° C.

To detect cultures that express an AGP-3 binding protein, the growth media of each well was removed and replaced with 100 μl DMEM containing 2% goat serum, 5% rabbit serum (Life Technologies, Gaithersburg, Md.) and 0.1 nM europium-labeled AGP-3 protein. Cells were incubated at RT for 1 hr. The cells were washed three times with 175 μl cold PBS, and then resuspended with 170 μl of Enhancer Solution (EG&G Wallac, Turku, Finland). The 96 well plates were then subject to analysis by Victor™ 1420 Multiplabel Counter (Wallac, Inc., Gaithersburg, Md.).

Using this approach, a total of approximately 300,000 independent RAJI cDNA clones were screened, represented by 3000 transfected pools of 100 clones each. Transfection and binding assay of each pool was performed in duplicates. Six wells were identified that contained cells which acquired the ability to be specifically decorated by the Europium-labeled AGP-3 protein. The positive signals ranged from 2-10 fold (Table 2). Five hundred bacteria colonies were picked for positive pool 13B4 and pool 13H11. The bacteria were cultured overnight. Plasmid DNA from each culture was prepared using the Qiagen Qiawell 96 Ultra Plasmid Kit (catalog #16191) following manufacturer's recommended procedures. Each plasmid prepreparation was transfected to 293 cells and examined for binding activity with europium-labeled AGP-3 as described above. Sixteen out of 500 clones of pool 13B4 were positive for AGP-3 binding, and 8 out of 500 clones of pool 13H11 were positive. Positive binding clones of both 13B11 and 13H4 pools were subject to sequence analysis. The positive binding clones from 13B11 and 13H4 pools encoded same gene, the latter has extra 7 basepairs at the N-terminus (FIG. 16).

AGP-3 Receptor DNA and Protein Sequence

The RAJI-13H4 clone isolated above contained an approximately 1.6 kb cDNA insert (FIG. 15), which was sequenced in both directions on an Applied Biosystems 373A automated DNA sequencer using primer-driven Taq dye-terminator reactions (Applied Biosystems) following the manufacturer's recommended procedures. The resulting nucleotide sequence obtained was compared to the DNA sequence database using the FASTA program (GCG, Univeristy of Wisconsin), and analyzed for the presence of long open reading frames (LORF's) using the “Six-way open reading frame” application (Frames) (GCG, Univeristy of Wisconsin). A LORF of 293 amino acid (aa) residues beginning at methionine was detected in the appropriate orientation, and was preceded by a 5′ untranslated region of about 17 bp and an in-frame stop codon upstream of the predicted start codon (FIG. 17). The RAJI-13B4 clone encodes a LORF of the same 293 amino acid residues, with 5′ 10 bp untranslated region and an in-frame stop codon. This indicates that the structure of the RAJI- plasmid is consistent with its ability to utilize the CMV promoter region to direct expression of a 293 aa gene product in mammalian cells.

The AGP-3 receptor contains a probably hydrophobic transmembrane domain that begins at a T166 and extends to L186. Based on this configuration relative to the methionine start codon, the AGP-3 receptor is predicted to be a type III transmembrane protein, with a N-terminal extracellular domain, a transmembrane region and a C-terminal intracellular domain. Unlike most other TNFR receptor family members, AGP-3 receptor contains two cysteine rich repeats within its N-terminal extracellular domain (FIG. 4).

The predicted AGP-3 receptor protein sequence was then compared to the existing database of known protein sequences using a modified version of the FASTA program (Pearson, Meth. Enzymol. 183, 63-98 (1990)). The amino acid sequence was also analyzed for the presence of specific motifs conserved in all known members of the tumor necrosis factor receptor (TNFR) superfamily using the sequence profile method of (Gribskov et al. (1987), Proc. Natl. Acad. Sci. USA 83, 4355-9), as modified by Luethy et al.(1994), Protein Sci. 3, 139-146.

### Expression of Human AGP-3 Receptor mRNA

Multiple human tissue northern blots (Clontech, Palo Alto, Calif.) were probed with a ³²P-dCTP labeled AGP-3 receptor restriction fragment to detect the size of the human transcript and to determine patterns of expression. Northern blots were prehybridized in 5×SSPE, 50% formamide, 5×Denhardt's solution, 0.5% SDS, and 100 μg/ml denatured salmon sperm DNA for 2-4 hr at 42° C. The blots were then hybridized in 5×SSPE, 50% formamide, 2×Denhardt's solution, 0.1% SDS, 100 μg/ml denatured salmon sperm DNA, and 5 ng/ml labeled probe for 18-24 hr at 42° C. The blots were then washed in 2×SSC for 10 min at RT, 1×SSC for 10 min at 50° C., then in 0.5×SSC for 10-15 minutes.

Using a probe derived from a 1.5 kb SalI NotI fragment of human AGP-3 receptor and hybridization under stringent conditions, a predominant mRNA species with a relative molecular mass of about 2.4 kb was detected in spleen, lymph nodes, skeletal muscle and heart (FIG. 20).

### Production of Recombinant AGP-3 Receptor Protein in Mammalian Cells

Expression construct was generated that direct synthesis of AGP-3 receptor extracellular domain fused with the Fc region of human. The following sets of oligonucleotide primers were used to PCR extracellular domain of human AGP-3 receptor (amino acids 1-166) was PCR amplified with the following set of oligonucleotide primers:

| | |
|---|---|
| 5′ TCT CCA AGC TTC CGA TCC TGA GTA ATG AGT GG -3′ | (SEQ ID NO: 50) |
| 5′ TCT CCG CGG CCG CGC TGT AGA CCA GGG CCA CCT G-3′ | (SEQ ID NO: 51) |

PCR reactions were carried in a volume of 50 μl with 1 unit of vent DNA polymerase (New England Biolabs) in 20 mM Tris-HCl pH 8.8, 10 mM KCl, 10 mM (NH₄)₂SO₄, 0.1% Triton-X100, 10 μM of each dNTP, 1 μM of each primer and 10 ng of ODAR cDNA template. Reactions were performed in 94° C. for 30 s, 55° C. for 30 s, and 72° C. for 1 min, for a total of 16 cycles. The PCR fragment was isolated by electrophoresis. The PCR fragment creates a Hind III restriction site at 5′ end and a Not I restriction site at 3′ end. The Hind III-Not I digested PCR fragment was then subcloned in-frame into a modified Fc/pCEP4 vector in front of the human IgG-γ1 heavy chain sequence as described previously in WO97/23614 and in Simonet et al. supra).

The construct was transfected into 293-EBNA-1 cells by calcium phosphate method as described (Ausubel et al. (1994), Curr. Prot. Mol. Biol. 1, 9.1.1-9.1.3. Both medium and cell lysates from transfected 293 cells were subject to western analysis with rabbit anti-human IgG Fc polyclonal antibody and subsequently with horseradish peroxidase linked anti-rabbit antibody (Amersham, Piscataway, N.J.). The extracellular domain of AGP-3 receptor fused with Fc domain was detected only in the cell lysates could be immunoprecipitated with AGP-3 protein. This finding supports that AGP-3 receptor is a type III transmembrane domain, which doesn't have an N-terminal signal peptide (FIG. 21). The receptor is directed to cell surface probably by an internal signal anchor sequence. Soluble receptor protein could be generated by grafting with an N-terminal signal peptide.

### Abbreviations

Abbreviations as used throughout this specification are defined as follows, unless otherwise defined in specific instances.

CDR complementarity determining region

dsDNA double-stranded DNA

EST expressed sequence tag

FCS fetal calf serum

ORF open reading frame

PBS phosphate-buffered saline

SDS sodium dodecyl sulfate

TNF tumor necrosis factor

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A composition of matter comprising the structure
(X¹)ₐ—F¹—(X²)_{b}
wherein:
F¹ is a vehicle;
X¹ and X² are each independently selected from —(L¹)_{c}—P¹—(L²)_{d}—P², —(L¹)_{c}—P¹—(L²)_{d}—P²—(L³)ₑ—P³, and —(L¹)_{c}—P¹—(L²)_{d}—P²—(L³)ₑ—P³—(L⁴)_{f}—P⁴
P¹, P², P³, and P⁴ are each independently selected from SEQ ID NOS: 45 and 46;
L¹, L², L³, and L⁴ are each independently linkers; and
a and b are each independently 0 or 1, provided that at least one of a and bis 1;
c, d, e, and f are each independently 0 or 1, provided that if P¹ is SEQ ID NO: 45 and P² is SEQ ID NO: 46, then d is 1;
and wherein said composition of matter does not comprise SEQ ID NO: 43.

2. The composition of matter of claim 1 of the formulae
X¹—F¹
or
F¹—X².

3. The composition of matter of claim 1 of the formula
F¹(L¹)_{c}P¹(L²)_{d}—P².

4. The composition of matter of claim 1 wherein F¹ is an Fc-region.

5. The composition of matter of claim 1 wherein F¹ is an IgG Fc domain.

6. The composition of matter of claim 1 wherein F¹ is an IgG1 Fc domain.

7. The polypeptide of claim 1, wherein F¹ is a water-soluble polymer or a carbohydrate.

8. The protein of claim 7, wherein the polymer is polyethylene glycol.

9. The protein of claim 7, wherein the carbohydrate is dextran.

10. A polypeptide of claim 1 capable of eliciting B cell growth, survival, or activation in mesenteric lymph nodes.

11. An isolated nucleic acid encoding a polypeptide of claim 1.

12. The nucleic acid of claim 11 including one or more codons preferred for Escherichia coli expression.

13. The nucleic acid of claim 11 having a detectable label attached thereto.

14. An expression vector comprising the nucleic acid of claim 11.

15. A host cell comprising the expression vector of claim 14.

16. The host cell of claim 15, wherein the cell is a prokaryotic cell.

17. The host cell of claim 16, wherein the cell is Escherichia coli.

18. A pharmaceutical composition comprising a therapeutically effective amount of a protein of claim 1 in a pharmaceutically acceptable carrier, adjuvant, solubilizer, stabilizer and/or anti-oxidant.

19. A method of modulating AGP-3-related activity in a mammal, which comprises administering a therapeutically effective amount of the composition of matter of claim 1.

20. The method of claim 22, wherein the AGP-3-related activity takes place in mesenteric lymph nodes.

21. A polypeptide comprising an antibody sequence in which one or more amino acids from antibody variable domains or CDR regions are replaced by sequences selected from SEQ ID NOS: 45 and 46.

22. The polypeptide of claim 21, wherein a first CDR region is replaced by SEQ ID NO: 45 and a second CDR region is replaced by SEQ ID NO: 46.

23. The polypeptide of claim 21, wherein all CDR regions are replaced by SEQ ID NO: 45.

24. An isolated nucleic acid encoding a polypeptide of claim 21.

25. The nucleic acid of claim 24 having a detectable label attached thereto.

26. An expression vector comprising the nucleic acid of claim 24.

27. A host cell comprising the expression vector of claim 26.

28. A pharmaceutical composition comprising a therapeutically effective amount of a polypeptide of claim 21 in a pharmaceutically acceptable carrier, adjuvant, solubilizer, stabilizer and/or anti-oxidant.

29. A method of modulating AGP-3-related activity in a mammal, which comprises administering a therapeutically effective amount of the composition of matter of claim 21.

30. The method of claim 29, wherein the AGP-3-related activity takes place in mesenteric lymph nodes.


SEQUENCE LISTING (52 sequences)

SEQ ID NO: 1 - DNA, 1173 bp, Homo sapiens
  CDS (143)..(997)
gaattcggcacgagctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaaaaaaaaaaagtagttaaaaaaaaaaaaaaaaaaaaaaaaaaaaaaaaaaaaaaaaaaaaaaaaaaactcggaggggg

SEQ ID NO: 2 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 3 - DNA, 1139 bp, Mus musculus
  CDS (52)..(978)
gaattcggcacgagctccaaaggcctagaccttcaaagtgctcctcgtggaatggatgagtctgcaaagaccctgccaccaccgtgcctctgtttttgctccgagaaaggagaagatatgaaagtgggatatgatcccatcactccgcagaaggaggagggtgcctggtttgggatctgcagggatggaaggctgctggctgctaccctcctgctggccctgttgtccagcagtttcacagcgatgtccttgtaccagttggctgccttgcaagcagacctgatgaacctgcgcatggagctgcagagctaccgaggttcagcaacaccagccgccgcgggtgctccagagttgaccgctggagtcaaactcctgacaccggcagctcctcgaccccacaactccagccgcggccacaggaacagacgcgctttccagggaccagaggaaacagaacaagatgtagacctctcagctcctcctgcaccatgcctgcctggatgccgccattctcaacatgatgataatggaatgaacctcagaaacatcattcaagactgtctgcagctgattgcagacagcgacacgccgactatacgaaaaggaacttacacatttgttccatggcttctcagctttaaaagaggaaatgccttggaggagaaagagaacaaaatagtggtgaggcaaacaggctatttcttcatctacagccaggttctatacacggaccccatctttgctatgggtcatgtcatccagaggaagaaagtacacgtctttggggacgagctgagcctggtgaccctgttccgatgtattcagaatatgcccaaaacactgcccaacaattcctgctacttggctggcatcgcgaggctggaagaaggagatgagattcagcttgcaattcctcgggagaatgcacagatttcacgcaacggagacgacaccttctttggtgccctaaaactgctgtaactcacttgctggagtgcgtgatccccttccctcgtcttctctgtacctccgagggagaaacagacgactggaaaaataaaagatggggaaagccgtcagcgaaagttttctcgtgacccgttgaatctgatccaaaccaggaaatataacagacagcc

SEQ ID NO: 4 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYLAGIARLEEGDEIQLAIPRENAQISRNGDDTFFGALKLL

SEQ ID NO: 5 - protein, 278 aa, Homo sapiens
  MUTAGEN (3, 5, 6, 7, 8, 9, 10, 11, 12, 14, 15, 17, 18, 20, 22,25, 26, 27, 28, 29, 30, 31, 32, 33, 34, 35, 37, 38, 39, 40, 41,42, 43, 44, 47, 50, 51, 52, 54, 55, 57, 60, 78, 79, 86, 87, 88,89, 90, 91, 116, 120, 121, 122, 125, 128, 135, 136, 147, 152, 155,179, 181, 182, 190, 197, 198, 199, 204, 231, 245, 252, 265, 266,)..(269): Xaa is absent or any amino acid residue
MDXSXXXXXXXXLXXCXXKXEXMKXXXXXXXXXXXEXXXXXXXXDGXLLAATLLLALLSXXXTXXSXYQXAALQXDLXXLRXELQXXXXXXXPAXAGAPXXTAGXKXXXPXAPXXXNSSXXXRNXRAXQGPEETXXQDCLQLIADSXTPTIXKGXYTFVPWLLSFKRGSALEEKENKIXVXXTGYFFIYXQVLYTDXXXAMGHXIQRKKVHVFGDELSLVTLFRCIQNMPXTLPNNSCYSAGIAXLEEGDEXQLAIPRENAQISXXGDXTFFGALKLL

SEQ ID NO: 6 - protein, 102 aa, Consensus
  MUTAGEN (1, 8, 10, 12, 15, 18, 21, 23, 25, 29, 39, 41, 44, 47,49, 51, 53, 55, 57, 60, 64, 66, 68, 70, 72, 79, 81, 84, 90, 92)..(94): Xaa is absent or any amino acid residue
XPAAHLTXPXLXWAXLSXGVXLXNXLVVXGLYFIYSQVXFXGQXCPXVXLXHXVXVXYPXLLSXTXCXWXSXYLGGVFXLXGDXLYVNVXSXFXTFFGLFKL

SEQ ID NO: 7 - protein, 143 aa, Homo sapiens
EKKELRKVAHLTGKSNSRSMPLEWEDTYGIVLLSGVKYKKGGLVLNETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 8 - protein, 143 aa, Mus musculus
EKKEPRSVAHLTGNPHSRSIPLEWEDTYGTALISGVKYKKGGLVINETGLYFVYSKVYFRGQSCNNQPINHKVYMRNSKYPEDLVLMEEKRLNYCTTGQIWAHSSYLGAVFNLTSADHLVYNISQLSLINFEESKTFFGLYKL

SEQ ID NO: 9 - protein, 143 aa, Rattus rattus
ETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 10 - protein, 146 aa, Homo sapiens
GDQNPQIAARVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 146 aa, Mus musculus
GDEDPQIAAHVVSEANSNAASVLQWAKKGYYTMKSNLVMLENGKQLTVKREGLYYVYTQVTFQSNREPSSQRPFIVGLWLKPSIGSERILLKAANTHSSSQLCEQQSVHLGGVFELQAGASVFVNVTEASQVIHRVGFSSFGLLKL

SEQ ID NO: 12 - protein, 144 aa, Homo sapiens
VTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 13 - protein, 147 aa, Mus musculus
LRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 14 - protein, 160 aa, Mus musculus
GKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 15 - protein, 160 aa, Homo sapiens
SKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 16 - protein, 166 aa, Homo sapiens
ERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 17 - protein, 172 aa, Mus musculus
GGRPQKVAAHITGITRRSNSALIPISKDGKTLGQKIESWESSRKGHSFLNHVLFRNGELVIEQEGLYYIYSQTYFRFQEAEDASKMVSKDKVRTKQLVQYIYKYTSYPDPIVLMKSARNSCWSRDAEYGLYSIYQGGLFELKKNDRIFVSVTNEHLMDLDQEASFFGAFLIN

SEQ ID NO: 18 - protein, 143 aa, Homo sapiens
RAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 19 - protein, 143 aa, Mus musculus
STPSKKSWAYLQVSKHLNNTKLSWNEDGTIHGLIYQDGNLIVQFPGLYFIVCQLQFLVQCSNHSVDLTLQLLINSKIKKQTLVTVCESGVQSKNIYQNLSQFLLHYLQVNSTISVRVDNFQYVDTNTFPLDNVLSVFLYSSSD

SEQ ID NO: 20 - protein, 163 aa, Homo sapiens
DLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 21 - protein, 159 aa, Mus musculus
DLNPELPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVG

SEQ ID NO: 22 - protein, 149 aa, Homo sapiens
AHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 23 - protein, 149 aa, Mus musculus
THGILKPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFAL

SEQ ID NO: 24 - protein, 152 aa, Homo sapiens
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 25 - protein, 29 aa, Artificial Sequence
  AGP-3 RELATED PROTEIN
  MUTAGEN (11, 16)..(19): Xaa is absent or any amino acid residue
QDCLQLIADSXTPTIXKGXYTFVPWLLSF

SEQ ID NO: 26 - protein, 25 aa, Artificial Sequence
  CONSENSUS
  MUTAGEN (5)..(5): Xaa is absent or any amino acid residue
AMGHXIQRKKVHVFGDELSLVTLFR

SEQ ID NO: 27 - protein, 142 aa, Artificial Sequence
  CONSENSUS
  MUTAGEN (43, 45, 46, 54, 61-63, 68, 95, 109, 116, 129,130)..(133): Xaa is absent or any amino acid residue
QDCLQLIADSXTPTIXKGXYTFVPWLLSFKRGXALEEKENKIXVXXTGYFFIYXQVLYTDXXXAMGHXIQRKKVHVFGDELSLVTLFRCIQNMPXTLPNNSCYSAGIAXLEEGDEXQLAIPRENAQISXXGDXTFFGALKLL

SEQ ID NO: 28 - DNA, 20 bp, Mus musculus
aattaaccctcactaaaggg

SEQ ID NO: 29 - DNA, 33 bp, Mus musculus
tctccctcgagatcacgcactccagcaagtgag

SEQ ID NO: 30 - DNA, 24 bp, Mus musculus
aacaggctatttcttcatctacag

SEQ ID NO: 31 - DNA, 25 bp, Mus musculus
ctcatcaatgtatcttatcatgtct

SEQ ID NO: 32 - DNA, 25 bp, Mus musculus
ctcatcaatgtatcttatcatgtct

SEQ ID NO: 33 - DNA, 20 bp, Mus musculus
agccgcggccacaggaacag

SEQ ID NO: 34 - DNA, 19 bp, Mus musculus
tggatgacatgacccatag

SEQ ID NO: 35 - protein, 7 aa, Homo sapiens
MNSRNKR

SEQ ID NO: 36 - DNA, 60 bp, Homo sapiens
atttgattctagaaggaggaataacatatgaacagccgtaataagcgtgccgttcagggt

SEQ ID NO: 37 - DNA, 45 bp, Homo sapiens
ccgcggatcctcgagttacagcagtttcaatgcaccaaaaaatgt

SEQ ID NO: 38 - protein, 17 aa, Homo sapiens
MDYKDDDDKKLNSRNKR

SEQ ID NO: 39 - DNA, 48 bp, Homo sapiens
gacgatgacaagaagcttaacagccgtaataagcgtgccgttcagggt

SEQ ID NO: 40 - protein, 151 aa, Mus musculus
QNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 41 - DNA, 1340 bp, Homo sapiens
  CDS (28)..(906)
gtcgacccacgcgtccgatcctgagtaatgagtggcctgggccggagcaggcgaggtggccggagccgtgtggaccaggaggagcgctttccacagggcctgtggacaggggtggctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggactggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagatgaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagcggccttggcattgtgtgtgtgcctgcccaggaggggggcccaggtgcataaatgggggtcagggagggaaaggaggagggagagagatggagaggaggggagagagaaagagaggtggggagaggggagagagatatgaggagagagagacagaggaggcagagagggagagaaacagaggagacagagagggagagagagacagagggagagagagacagagaggaagagaggcagagagggaaagaggcagagaaggaaagagacaggcagagaaggagagaggcagagagggagagaggcagagagggagagaggcagagagacagagagggagagagggacagagagagatagagcaggaggtcggggcactctgagtcccagttcccagtgcagctgtaggtcgtcatcacctaaccacacgtgcaataaagtcctcgtgcctgctgctcacagcccccgagagcccctcctcctgg

SEQ ID NO: 42 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKMRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 43 - protein, 291 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVAVYSTLGLCLCAVLCCFLVAVACFLKMRGDPCSCQPRSRPRQSPAKSSQDHAMEAGSPVSTSPEPVETCSFCFPECRAPTQESAVTPGTPDTCAGRWGCHTRTTVLQPCPHIPDSGLGIVCGPAQEGGPGA

SEQ ID NO: 44 - protein, 32 aa, Homo sapiens
MSGLGRSRRGGRSRVDQEERFPQGLWTGVAMR

SEQ ID NO: 45 - protein, 37 aa, Homo sapiens
SCPEEQYWDPLLGTCMSCKTICNHQSQRTCAAFCRSL

SEQ ID NO: 46 - protein, 38 aa, Homo sapiens
SCRKEQGKFYDHLLRDCISCASICGQHPKQCAYFCENK

SEQ ID NO: 47 - protein, 57 aa, Homo sapiens
LRSPVNLPPELRRQRSGEVENNSDNSGRYQGLEHRGSEASPALPGLKLSADQVAVYS

SEQ ID NO: 48 - protein, 21 aa, Homo sapiens
TLGLCLCAVLCCFLVAVACFL

SEQ ID NO: 49 - protein, 106 aa, Homo sapiens
KMRGDPCSCQPRSRPRQSPAKSSQDHAMEAGSPVSTSPEPVETCSFCFPECRAPTQESAVTPGTPDTCAGRWGCHTRTTVLQPCPHIPDSGLGIVCGPAQEGGPGA

SEQ ID NO: 50 - DNA, 32 bp, Homo sapiens
tctccaagcttccgatcctgagtaatgagtgg

SEQ ID NO: 51 - DNA, 34 bp, Homo sapiens
tctccgcggccgcgctgtagaccagggccacctg

SEQ ID NO: 52 - protein, 6 aa, Homo sapiens
GALKLL